(19) United States
(12) Patent Application Publication
(10) Pub. No.: US 2023/0273197 A1
(43) Pub. Date: Aug. 31, 2023

(54) CARRIER SYSTEM AND METHOD

(71) Applicant: CAMBRIDGE ENTERPRISE LIMITED, Cambridge (GB)

(72) Inventors: Jeroen Verheyen, Cambridge (GB); Tarun Vemulkar, Cambridge (GB); Russell Cowburn, Cambridge (GB)

(21) Appl. No.: 17/923,594

(22) PCT Filed: May 6, 2021
(86) PCT No.: PCT/GB2021/051101

§ 371 Date: Nov. 6, 2022

(30) Foreign Application Priority Data
May 7, 2020 (GB) 2006800.3

(51) Int. Cl. G01N 33/543

(52) U.S. Cl. CPC G01N 33/54326

(57) ABSTRACT

A carrier system (100) provides a carrier or carriers (12) for carrying assay samples in an assay. The carrier(s) are secured to a substrate (10) by a release layer (14). The carrier(s) are suitable for receiving an assay sample, and the release layer is configured to release the carrier(s) from the substrate in the presence of a biocompatible aqueous solution. To perform an assay a biocompatible aqueous solution, in which the assay sample is usually suspended, is supplied to the carrier system. The assay sample is received by the carrier(s) and the release layer is activated by the biocompatible aqueous solution to release the carrier.

The invention relates to a carrier system and method, and in particular to a carrier system for an assay, and to methods of manufacturing the carrier system and performing an assay using the carrier system.

### BACKGROUND ART

An assay is an investigative procedure for qualitatively or quantitatively measuring the presence, amount or activity of an analyte in an assay sample. Often, the assay sample is a cell or a cell culture and the analyte is a protein or gene sequence of that cell or cell culture, but analyses may more generally include metabolites, peptides, proteins, nucleic acids, extracellular vesicles, organelles, cells, or tissues.

Often, the assay sample is exposed to a reagent prior to a measuring step. In fields such as biology and pharmacology, it is common for assay samples to be exposed to a large number of different reagents. For example, if the analyte is a protein or a gene sequence of a cell culture serving as a disease model, different samples of the cell culture may be exposed to a large number of different drug candidates. The interaction between the assay sample and the reagent typically occurs in a well plate. The measuring step may take place in the well plate or elsewhere. In some cases, the measuring step can take place in flow conditions, for example, in a flow cytometer.

Some biological assay samples, such as certain types of cells, may be suspended in an aqueous solution. However, there are many biological assay samples which cannot easily be suspended in solution and which are most viable when attached to a surface, such as adherent cells. Assays of such samples, including the step of measuring, typically have to be performed while the assay sample is attached to the bottom of a well plate. In such assays, the assay process may be disadvantageously limited by the fact that an assay sample attached to the bottom of a well plate cannot easily be transferred to another container without stripping it from the well plate. This may damage the sample.

Measuring an assay sample attached to a well plate is inefficient. The assay sample is typically attached or seeded across the entire well plate but only one or two percent of the assay sample is actually imaged or measured. This is a particular problem when the assay sample is a scarce resource, for example cells that are particularly difficult to culture or primary cells such as cells collected during a biopsy of a tumour.

Adherent cells are an example of an assay sample that is most viable when attached to a surface. There is a strong interest in assaying adherent cells under flow conditions using, for example, a flow cytometer. However, this requires stripping of the adherent cells from their attached state prior to the assay. This may bias the assay being performed because the adherent cells may be damaged or because adherent cells forced into suspension may not accurately represent the normal biological state of the adherent cell. The significant extent of this problem is illustrated by the fact that in conventional assays, after stripping an adherent cell from a substrate, it is usual to wait for 12 to 48 hours for the cell to return to a normal or undisturbed state.

Another problem of performing an assay on an assay sample that is most viable when attached to a surface, or which tends to agglomerate on surfaces, is that such assay samples may have a tendency to clog channels in flow cytometry apparatus.

### SUMMARY OF INVENTION

The invention provides a carrier system for an assay, a method of manufacturing a carrier system for an assay and a method of using a carrier system for an assay, as defined in the appended independent claims to which reference should now be made. Preferred or advantageous features of the invention are set out in the dependent claims.

A first aspect of the invention may thus advantageously provide a carrier system for an assay, comprising a carrier or particle for carrying an assay sample. The carrier is secured to a substrate by a release layer. The carrier is advantageously suitable for receiving an assay sample, and the release layer configured in use to release the carrier from the substrate in the presence of a biocompatible aqueous solution. For example, the release layer may be activated, to release the carrier, by the biocompatible aqueous solution. The release layer, when activated, may thus release the carrier from the substrate.

An assay sample for introduction into an array system may commonly be suspended in a biocompatible aqueous solution. When a user of the carrier system wants to perform an assay, contact between the biocompatible aqueous solution carrying the assay sample may then automatically release the carrier into the solution.

Preferably, the carrier system may thus be contacted with the biocompatible aqueous solution carrying the assay sample so that the assay sample (for example an adherent cell) may attach to the carrier, before the carrier is released into solution, carrying the assay sample.

In a preferred embodiment, the assay sample may be an adherent cell and the carrier, or particle, may be of a suitable size for the cell to adhere to the carrier. For example, as described in more detail below, the carrier may have a planar upper surface of lateral dimension in the range of 5 to 200 micrometres, suitable for an adherent cell to attach to. The assay sample may then be carried through the assay on the carrier. In a particularly preferred embodiment, the carrier may be magnetic so that it, and the assay sample that it carries, may be directed through the assay by the application of an external magnetic field.

Aspects of the invention are described herein with reference to a carrier or particle secured to a substrate, but in a typical application the carrier may be one of many carriers or particles secured to the same substrate and used for example to perform a multi-channel assay. In a typical embodiment of the invention, therefore, a substrate may carry many carriers, such as more than 10, 100, or 500 carriers, up to as many as 1000, 5000 or 10,000 or more. As described below, each carrier may be individually identifiable, or subgroups of the carriers may be identifiable, by way of markings such as barcodes carried by the carriers for use in a multi-channel assay. Multiple carriers, or assay-sample carriers, secured to a substrate may be termed a carrier array or particle array.

For the sake of simplicity, however, embodiments of the invention described herein will usually be described with reference to only one carrier.

In a preferred embodiment, an assay sample may be introduced to the carrier system so that the assay sample may be received by, or onto, the carrier. The assay sample may be a biochemical substance or a cell of an organism or an organic sample. Preferably, the assay sample may be an adherent cell. The assay sample received on the carrier may attach, adhere, seed onto or bind to a surface of the carrier, preferably before the carrier is released from the substrate. An assay may then be performed on the assay sample received on, and carried by, the released carrier. The assay may involve measuring the presence, amount or activity of an analyte of an assay sample. When the assay sample is a cell, the analyte may, for example, be a protein or gene sequence of that cell.

An assay sample received on a carrier may be convenient to manipulate and transport. This may be advantageous when performing an assay, for example in order to move the assay sample to a well plate, to expose the assay sample to a reagent or to position the assay sample with respect to a detector in a measuring step.

In a further embodiment of the invention, the receiving of an assay sample onto a carrier may be used to conveniently store assay samples for use in a future assay.

A conventional, prior-art, method of storing cells for an assay may involve:
Stripping the adherent cells (e.g. chemically) to make a single cell suspension in culture medium.
Concentrating the cells to about 1 M cells per mL (done with a centrifuge)
Complementing the culture medium with 5-20% DMSO (dimethyl sulfoxide) or equivalent.
This mixture is then added to freezing tubes (i.e. cryogenic storage vials)
These are then slowly cooled down to -70C at 1 C per minute (typically this is done by immersing the vials in isopropanol and placing that in a -70C freezer)
After that, the vials are stored at -70C (either in a freezer or in liquid nitrogen)
When thawing, the frozen vial is placed in a 37C warm water bath until thawed.
Fresh medium is then added, cells are spun down, and fresh medium is added again.
Cells are then seeded onto a flask or plate or dish for an assay to be performed.

Using an embodiment of the present invention, this method may be significantly improved as follows:
Attach the adherent cells to carriers attached to a substrate, as described herein.
Optionally, release the carriers from the substrate into liquid as described herein.
Alternately the carriers may be retained on the substrate.
Cells would then either be cultured for 1-2 days or frozen directly (the former is preferred to maintain healthy cells).
The substrate carrying the carriers, or the free carriers, loaded with or carrying the cells, are in a medium which is refreshed with medium containing 5-20% DMSO, making sure there is about 1 M cells per mL of liquid. Either a centrifuge or gravity may be used to concentrate the carriers as the medium is refreshed. Alternatively, if magnetic carriers are used, as described herein, the carriers may be magnetically pulled down or held down as the medium is refreshed.
Then the protocol would go as in conventional cell freezing, except that the cells are received on carriers ready for use in an assay.

This embodiment of the invention provides numerous benefits:
Cells do not need to be stripped prior to freezing
Cells do not need to be seeded or attached to any substrate after freezing
It is possible to store live, adhered cells.
This is useful if researchers would want to do an experiment (which needs adherent cells), but would like to store some of the assayed samples for later analysis or for additional experiments.
This is useful when people culture a large batch of cells on which they want to do multiple experiments over a long time. They may then attach the whole batch, or a plurality of the cells, to the carriers, which are then frozen. They can then thaw a portion or all of the cells when they want to do an experiment. This ensures minimal inter-experimental variability and bias. What people need to do conventionally is continuously grow cells (over multiple passages, i.e. cycles of stripping and re-seeding when cells are overgrown in culture), but mutations and changes accumulate over these different passaging steps. The other known solution is to always thaw a fresh batch prior to an experiment. This is however not only labour intensive (because you need to thaw, grow for 2 days, strip and re-seed), but also introduces variability and potentially bias.
The embodiment of the invention is useful when people want to do a quick assay. This approach makes sure that people do not need to do the “thaw, grow for 2 days, strip and re-seed” step prior to an assay. In this situation, cells are thawed and the experiment/assay can either proceed directly or after 1-2 days of culturing (without the strip and re-seed step).

Conventionally, assay-ready cells are available, but these cells are still suspended in liquid and need to be attached to a substrate prior to assaying. The embodiment of the invention ensures that these assay-ready cells are all frozen in the same “state” (e.g. how many times they have been passaged, what cell cycle step they are in, making sure that they have no artefacts such as mutations or metabolic issues). Thus, in an embodiment of the invention assay-ready cells on carriers may be sold, for example in a frozen state, ready for use. Further, as described herein, the assay-ready cells may be on carriers which are individually identified by, for example, barcodes or other readable markings on each carrier.

In a preferred embodiment of the carrier system of the invention, the release layer may be activatable in the presence of an assay-sample solution or assay-sample medium for containing or carrying an assay sample. The assay-sample solution may for example comprise a biocompatible aqueous solution and an assay sample. Therefore, the single act of introducing the assay-sample solution to the carrier system may result in the assay sample being received by the carrier and the carrier being released from the substrate.

Many assay samples must be maintained and transported in a biocompatible aqueous solution in order to maintain viability. Therefore the provision of carriers, for receiving an assay sample, secured to a substrate by a release layer configured to release the carriers in the presence of a biocompatible aqueous solution is particularly convenient. The biocompatible aqueous solution may for example be non-cytotoxic.

In a preferred embodiment, the release layer may be configured such that, following activation of the release layer, the biocompatible aqueous solution remains biocompatible. The release layer may preferably not release non-biologically-compatible or toxic components into the biocompatible aqueous solution. Such components might otherwise render the biocompatible aqueous solution toxic to the assay sample. More generally, the release layer may preferably not release into the biocompatible aqueous solution any materials or components that may modify or change the assay sample in any way. Such a release layer may advantageously not impact the results of an assay performed using the carrier system. The release layer may for example be formed of a biocompatible material.

In a preferred embodiment, the assay sample is one that is at its most viable when received on a surface. An example of such an assay sample may be an adherent cell. Once such an assay sample has been received by the carrier, or onto a surface of the carrier, and the carrier has been released from the substrate, the assay sample may remain on the carrier rather than moving onto another surface. This may mean that such assay samples, received on and carried by released carriers, are convenient to manipulate and transport. Furthermore, such assay samples may remain in a representative biological state while the assay is being performed. This may reduce any bias in an assay of such assay samples. In particular, there may be no need to force such assay samples into suspension via mechanical or chemical means, as would be required in prior art methods. The agglomeration of such assay samples may also advantageously be reduced.

Assay samples received on carriers embodying the invention may advantageously be measured for example using a flow cytometer or fluorescent microscopy.

In a preferred embodiment, the release layer may comprise a material that is water-activatable, in other words a material that releases the carrier or particle in the presence of water, such as water in a biocompatible aqueous solution. The water-activatable material may be a water-soluble material. The water-soluble material may dissolve in the presence of water to release the carrier. The release layer may or may not completely dissolve. The release layer may only dissolve to such an extent that the bond between the release layer and the carrier is weakened and the carrier is released from the substrate.

Alternatively, or additionally, the release layer may comprise a material having other properties that change in the presence of water or an aqueous solution. For example, the adhesion of the material of the release layer may be reduced in the presence of water or an aqueous solution. As with water-soluble release layers, these release layers may have the advantage that the carrier may be released from the substrate in the presence of water, but preferably without adding any components of the release layer into the solution where they may affect the assay sample.

In a preferred embodiment as described in more detail later in this document, the manufacture of a carrier system may comprise a lithographic process. The release layer may then be formed of a material suitable for or compatible with the required lithographic process. The release layer may be applied to the substrate prior to at least some of the steps of the lithographic process, and the carriers then formed on top of the release layer. In order to be suitable for lithographic process, the release layer may advantageously not be affected by the processes and chemicals used in the lithographic process.

In a preferred embodiment, the release layer may comprise a material that is not activatable in a non-aqueous solvent such as ethanol. As described in more detail later in this document, the method of manufacturing the carrier system may comprise applying to the carrier a coating adapted for receiving an assay sample. This process may advantageously be carried out while the carrier is secured to the substrate by the release layer. The coating may for example be applied by immersing the carrier system in a solution for forming or depositing the coating, which may be a polymer, for a period of time such as between 10 and 120 minutes. Such solutions commonly comprise non-aqueous solvents such as ethanol and so advantageously the release layer may be unaffected, or may not be activated, by such solvents so that the release layer may retain the carrier secured to the substrate while the carrier system is exposed to the non-aqueous solvent. In particular, the release layer advantageously may not release the carrier when exposed to the non-aqueous solvent for the length of time, and under the conditions required, during manufacture of the carrier.

Alternatively or additionally, the carrier system may undergo a sterilization process while the carrier is secured to the substrate by the release layer. The sterilization may be part of the manufacturing process or in preparation of the carrier system for performing an assay. Sterilization advantageously eliminates unwanted forms of life or biological agents that might otherwise adversely interfere with assays performed using the carrier system. Sterilization may comprise immersing the carrier system in a non-aqueous sterilization liquid such as ethanol. The sterilization liquid may comprise pure ethanol. The carrier system may be immersed in the sterilization liquid for up to 10, 15, 20 or 25 minutes, typically up to a maximum of about 30 minutes or 1 hour. If the release layer comprises a material that is non-soluble in a non-aqueous solvent such as ethanol, the release layer may advantageously retain the carrier secured to the substrate while the carrier system is immersed in non-aqueous solvent. In particular, the release layer advantageously may not release the carrier when exposed to the non-aqueous solvent for the length of time, and under the conditions required, for sterilization of the carrier.

The release layer may comprise a sugar. Sugars are examples of materials that may be biocompatible. A release layer comprising a sugar may release the carrier in the presence of a biocompatible aqueous solution such as an aqueous solution. Sugars may be water-soluble. Preferably, the release layer may comprise a sugar such as a dextran. A release layer comprising a dextran may be biocompatible. In the manufacture of the carrier system, the release layer may be applied to the substrate using a spin-coating technique. A release layer comprising a sugar and, particularly, a dextran, may advantageously be suitable for spin-coating.

Dextrans may comprise polymer molecules of a range of lengths, typically from about 3 to 2000 kDa or more. A solution of any dextran may be spin coated to form the release layer. A dextran of 70 kDa has been found to provide a release layer activation time of between 1 and 10 minutes. Larger dextran molecules, in the range 5000 to 40000 kDa are less soluble in the biocompatible aqueous solution and may be used to provide longer release times. Therefore, smaller dextran molecules in the range of 3 kDa or 50 kDa to 500 kDa or 2000 kDa may be used to provide shorter release times, and larger dextrans such as in the range of 2000 kDa or 3000 kDa or 5000 kDa to 6000 kDa or 10000 kDa or 40000 kDa may be used to provide longer release times.

A release layer comprising a sugar, such as a dextran, may be suitable for lithographic processes, and may not be activatable in non-aqueous solvents such as solvents containing ethanol, or in pure ethanol.

The parameters of the release layer, such as its material(s), structure and thickness, may be selected or designed depending on the desired time taken for the carrier to be released following the introduction of a biocompatible aqueous solution to the carrier system. In a preferred embodiment, the parameters of the release layer may be selected to release the carrier at a time after which the assay sample is likely to have been received on the carrier. Receiving the assay sample on the carrier while the carrier is in contact with the substrate may ensure that the assay sample is received only by an exposed surface of the carrier. As only one surface of the carrier may typically be measured during an assay, this may advantageously ensure that a greater proportion of the assay sample is measured and reduces or eliminates wastage of the assay sample. This is particularly advantageous when the assay sample is a scarce resource, for example cells that are particularly difficult to culture or primary cells such as cells collected during a biopsy.

A release layer comprising a sugar such as a dextran may release the carrier relatively quickly in the presence of a biocompatible aqueous solution, such as in 5 or 10 seconds or less. Some assay samples may typically take much longer than five seconds to be received on the carrier following the introduction of the assay sample to the carrier system. For example, it may take an adherent cell three hours or more to be received on the carrier. Therefore, the release layer may comprise materials having a longer release time.

The release layer may comprise a polyvinyl alcohol (PVA). The release time of a release layer comprising polyvinyl alcohol may be much longer than the release time of a release layer comprising dextran. A release layer comprising polyvinyl alcohol may be configured to release the carrier about 1, 2, 3, 6 or 9 hours, up to as long as 12 hours or more, after the introduction of a biocompatible aqueous solution and an assay sample to the carrier system. A release layer comprising polyvinyl alcohol may therefore be designed to release the carrier a suitable time after the introduction of biocompatible aqueous solution such that an assay sample, such as an adherent cell, has been received on the carrier.

Like sugars, polyvinyl alcohols are materials that may be considered to be biocompatible. A release layer comprising a polyvinyl alcohol may be water-soluble, suitable for spin-coating and compatible with lithographic processes. A release layer comprising polyvinyl alcohol may not be activatable in non-aqueous solvents such as solvents containing ethanol, or in pure ethanol.

Other materials may be used to form the release layer, such as PLGA (PLG or poly(lactic-co-glycolic acid)) or a poly(acrylic acid) (PAA). The structure of the release layer may also be formed from multiple materials, such as layer-by-layer deposited polymers and/or sugars.As described above, manufacturing a carrier system embodying the invention may include a step in which a coating adapted for receiving an assay sample is applied to the carrier(s). Alternatively or additionally, the carrier system may undergo sterilisation. As described above, each of these processes may be performed using a non-aqueous solvent such as ethanol and the carrier may advantageously remain secured to the substrate in the presence of the non-aqueous solvent.

Alternatively, in some embodiments, the materials, structure and thickness of the release layer may be selected to allow for the use of an aqueous solution during manufacture, for example in the application of a coating or during a sterilization process. If the parameters of the release layer are selected so that the carrier is only released after a period that is greater than the cumulative time that the carrier system is immersed in aqueous solutions for either or both of the above processes, then these processes may be carried out and the carrier may remain secured to the substrate. It should also be borne in mind that the release layer should still provide time, when an assay is to be performed, for an assay sample to be received on the carrier while the carrier is secured by the release layer to the substrate.

In general, the materials and other properties, such as the thickness and structure, of the release layer may be varied or predetermined, in order to achieve a desired release time.

In a preferred embodiment, the carrier may comprise a magnetic material. This may have a number of advantages during an assay. When an external magnetic field is applied, the carrier may have a sufficient magnetic moment for the external field to apply a desired force to the carrier.

For example, the force may be applied to retain the carrier in contact with the substrate even after the release layer has otherwise released the carrier. As described above, it may be advantageous for the assay sample to be received by the carrier while the carrier is in contact with the substrate. By applying an external field to retain the carrier in contact with the substrate even after the release layer has released the carrier, the carrier can be held in place on the substrate for any length of time. This may, for example, remove the need to match the release time of the release layer with the typical time taken for a particular assay sample to be received by the carrier, or it may allow a carrier which is retained by a release layer designed to have a particular release time to receive assay samples which require any length of time longer than that release time to be received on the carrier. In other words, a release layer having an activation time that is less than the typical time taken for the particular assay sample to be received by the carrier may be used.

Similarly, the force applied by the external field is advantageously sufficient to move or drive the carrier through a solution during an assay, while carrying the assay sample. If the sample is a cell, for example, the force should be sufficient to move the carrier and the cell.

The carrier may be lithographically defined or fabricated. A lithographically defined carrier may advantageously have a high aspect ratio. Such a carrier may advantageously have a large surface area for receiving an assay sample relative to the volume of the carrier. In a preferred embodiment, the carriers may conveniently be lithographically defined or fabricated on or over the release layer of the carrier system.

The carrier may comprise a photoresist layer which may be an artefact of a lithographic process used to define or fabricate the carrier. The photoresist layer may advantageously comprise a material or materials that are not soluble in water.

During manufacture of the carrier, a photoresist layer may be patterned by exposing it to radiation and washing away or dissolving regions of the photoresist layer in a solvent. In lithographic processes, photoresists are typically water-soluble and the solvent is an aqueous solvent. In embodiments of the invention, a photoresist that is not soluble in water is preferred. The step of washing away the photoresist may then advantageously involve a solvent other than a water-based solvent. Therefore, the release layer may advantageously be unaffected by the washing process.

The photoresist layer may comprise an SU-8 photoresist. An SU-8 photoresist may be soluble in a solvent comprising, for example, propylene glycol methyl ether acetate, gamma-butyrolactone or cyclopentanone. The SU-8 photoresist may advantageously not be soluble in a biocompatible aqueous solution. An SU-8 photoresist may therefore be biocompatible, if present in an assay.

In a preferred embodiment, a surface of the carrier may be adapted or modified for receiving an assay sample. Such an adaption may advantageously improve the efficiency or rate at which an assay sample is received by that surface and so may reduce the time for an assay sample to be received on the carrier and increase the strength of bonding or attraction between the assay sample and the carrier so that the assay sample is advantageously securely bonded to the carrier. This may also reduce any tendency for the assay sample to migrate or move away from the carrier during an assay. The surface of the carrier may be adapted for receiving adherent cells. The adapted surface may be a surface of the carrier that is exposed when the carrier is secured to the substrate.

The surface of the carrier may comprise a coating adapted for receiving an assay sample. Carriers comprising such a coating may be referred to as bio-functionalized. The coating may be adapted for receiving adherent cells as an assay sample. The coating may be a charged coating. This may be particularly preferable when the assay sample is inherently charged. For example, cells typically have a membrane potential of between negative 40 and negative 80 millivolts. Providing a coating defining a surface having a positive charge may encourage attachment of the assay sample onto the charged surface of the carrier. It has been found that providing a charged surface, and preferably a positively charged surface, may advantageously lead to more effective cell adhesion. The coating may comprise a charged polymer.

In some embodiments, the coating is applied on a gold layer of the carrier in the form of a gold cap.

The gold cap of the carrier may only be on one side or surface of the carrier. In particular, the gold cap may only be on a surface of the carrier that is exposed while the carrier is secured to the substrate. This ensures that only one surface of the carrier is bio-functionalized and so only one surface of the carrier tends to receive the assay sample. The advantages of only receiving the assay sample on one surface of the carrier are described herein. In some embodiments, the carrier may have been fabricated using lithographic processes. Lithographic processes may be particularly suitable for fabricating a carrier with only a specific surface comprising a gold cap.

The polymer (charged polymer) may be covalently bound to the gold cap. The surface adapted for receiving the assay sample may comprise a gold cap layer on to which a polymer is covalently bonded by a thiol group. This advantageously ensures that each polymer adsorbed onto the surface has the same orientation. Alternatively, the polymer may be adsorbed onto the gold cap of the carrier by Van der Waals forces.

The charged polymer may be a polymer comprising a positive-charge-carrying group. The polymer may be a polyornithine or a poly-d-lysine. The polymer may be a polyelectrolyte.

Alternatively or additionally, the coating may comprise a plurality of ligands. The plurality of ligands may comprise antibodies. When the assay sample of the assay is a cell, the plurality of ligands may comprise antibodies that specifically bind to cell receptors such as integrins.

When the assay sample is a cell, the coating may alternatively or additionally comprise an extracellular matrix protein. The extracellular matrix protein may be a protein selected to increase or enhance cell adhesion. The protein may be a collagen, laminin or Matigel, a protein mixture secreted by Engelbreth-Holm-Swarm (EHS) mouse sarcoma cells.

A surface of the carrier may comprise a physical structure adapted for receiving adherent cells. The physical structure or topography of a surface can have an effect on the degree to which adherent cells adhere or seed onto that surface. Cell adhesion may advantageously be increased by increasing the surface area of the surface of the carrier. The surface area may be increased by increasing the roughness of the surface. For example, the surface may have been etched using an ion beam.

The hardness of a surface may also have an effect on the rate or efficiency to which adherent cells are received on that surface of the carrier. Cell adhesion is closely related to the Young’s modulus of the surface. Therefore, the hardness, or Young’s modulus, of the surface of the carrier may be tuned to optimize the adhesion of the cell that is the assay sample.

To summarise, the steps in an assay according to a typical embodiment of the invention, in an example where the assay sample comprises adherent cells, may include the following;
1. Carrier array placed in a well plate, flask, or any other cell culture vessel.
2. Cells that have been stripped (single cells) are mixed with culture medium and loaded onto the carrier array.
3. The cells will settle down and attach to the carriers (typically minimum 2-3 h)
4. The aqueous medium slowly dissolves the sacrificial (release) layer.
5. The carriers with attached cells are released into liquid.
6. Carriers with cells can be moved, assayed, measured, stored, etc.

As described above, the carrier may comprise a magnetic material. When an external field is applied, the carrier may have a sufficient magnetic moment for the external field to apply a desired force to the carrier. The desired force may depend on the application of the carrier. In practical implementations, the applied external field is typically less than 2 T, or 1 T or 0.5 T, and may typically be greater than 0.05 T, or 0.1 T or 0.25 T. A carrier comprising a magnetic material may be steerable by the application of an external magnetic field. For example, the carrier may be steerable within a fluid medium. The fluid medium may be the biocompatible aqueous solution introduced to the carrier system to release the carrier. The magnetic moment may be due to magnetization of the carrier itself, or it may be induced in the carrier by the external field. It is important, however, for the carrier to contain sufficient magnetic material to enable the desired force to be generated. The magnetic moment that can be generated by an external field applied to a carrier may depend on the total volume V of magnetic material in the carrier multiplied by that material’s magnetization Ms. Thus, the value of V.Ms for a carrier embodying the invention is preferably greater than a predetermined value, such as 10-18 J/T or 5×10-18 J/T or 10-17 J/T.

The magnetic material may comprise a material selected for example from metals or metallic alloys such as Fe, Co, Ni, CoFe, CoFeB, FePt, CoNi and NiFe.

The external field may be applied to orientate the carrier, and the assay sample received on the carrier, with respect to a detector. This may advantageously improve the consistency and quality of results from an assay performed with such carriers as the carrier may be placed in the optimum position and angle with respect to the detector. The ability to orientate the assay sample received on the carrier is particularly advantageous when the carriers are in suspension, for example, in an aqueous solution and may be particularly beneficial in microfluidic or flow cytometry applications.

The structure of the magnetic carriers enables a number of advantages in an assay, such as enabling the changing of the liquid in which the carriers and assay samples are supported, sorting specific carriers (e.g. after measurement for collection), the settling of carriers to the bottom of a well or flask with the face of the carriers carrying the assay samples face-up (so that the assay samples are not pressed between carrier and well).

A preferred embodiment of an imaging assay using the magnetic carriers, for an assay sample comprising cells, may then comprise steps such as the following:
Assay sample attached to carriers, while using magnetic field to hold carriers on substrate after release layer activated
Assay sample (on carriers) moved to a fresh container or well (to remove unattached cells (i.e. any cells not attached to carriers) and refresh the culture medium)
Magnetically pull down the carriers with cells to the bottom of the container with cells facing up (for incubation or cell growth).
Add assay reagents (e.g. a drug)
Remove liquid (using magnetic field to keep carriers in place)
Add various assaying liquids sequentially (e.g. wash buffers, labelled antibodies) (using magnetic field to hold carriers in place as liquids are changed)
Flip over the carriers (using magnetic field) for microscope imaging through the bottom of the well.

In an alternative embodiment, using a flow-based assay (again described in the context of an assay sample comprising cells) the use of carriers embodying the invention may provide further advantages, such as not aggregating (clogging), allowing the imaging of groups of cells or single cells on carriers that pass detectors, and allowing measurement using either fluorescent intensity (spectrometry) or by taking full images of cells on carriers.

In an embodiment of the invention, the carrier or particle may comprise a layered structure between a top surface of the carrier and an opposed bottom surface of the carrier, the layers including one or more magnetized layers; in which the ratio of a lateral dimension of the one or more magnetized layers to a thickness or aggregate thickness of the magnetized layer or layers is preferably greater than 500. Carriers having such a structure may have a low stray field at the surfaces of the carrier. This may advantageously mean that when a plurality of carriers having the same structure are provided the carriers may not interact with another. In particular, the carriers may not aggregate or clump. This may be the case whether or not the carriers are magnetically remanent.

The carrier may further comprise a non-magnetic layer, which may advantageously provide mechanical support to the magnetic layer, and may determine physical characteristics of the carrier such as its mechanical properties and its density. The non-magnetic layer may provide a suitable substrate for the magnetized layer. The non-magnetic layer may thus advantageously comprise a material selected from Al, Ta, Pt, Pd, Ru, Au, Cu, W, MgO, Cr, Ti, Si, Ir, SiO₂, SiO, Sn, Ag, polymers and plastics, alloys of these materials, and composites or mixtures comprising these materials.

In a preferred embodiment, the carrier may comprise readable information, such as a readable code selected from a barcode or a 2D code. This may allow the carrier to be identified remotely by reading the information, for example with a camera and suitable software. A multi-channel assay may be performed by providing a plurality of carriers in which each carrier carries readable information.

In a preferred embodiment, the readable information may be used to identify the assay sample received on the carrier. In a multi-channel assay, more than one type of assay sample may be analysed at once. Where the assay sample is a cell, more than one different type of cell may be analysed at once. The readable code may indicate the assay sample type on a particular carrier. The assay may be performed using a plurality of differently coded carriers, with the differently coded carriers carrying different types of assay sample. Following the assay samples being received on the carriers, the carriers having different types of assay sample may be pooled. For example, a plurality of carriers, each comprising (carrying) different types of cells, may be pooled or mixed in a well plate containing a particular drug in order to expose the various cells to that drug. The identification of carriers using readable information in this way may advantageously provide a multiplexed platform in which the carriers, and therefore the assay samples received on the carriers, can be accurately distinguished from each other.

It may be that assay samples of a particular type are received by carriers having identical readable information. In that case, the readable information need only distinguish between carriers carrying different assay sample types. In other words, the number of channels, or the plex, of the assay equals the number of individual assay sample types used in the assay. However, it may be advantageous to provide each carrier with unique readable information. A subset of the carriers may comprise unique readable information relating to a particular assay sample type. The unique readable information of the carriers in the subset may also be used to identify the test conditions experienced by each assay sample in the subset during the assay, for example, the particular drug that the assay sample is exposed to during the course of the assay. In this case, the readable information needs to be capable of distinguishing between every carrier of the plurality of carriers of the carrier system. In other words, the plex of the assay may equal the number of carriers used in the assay.

The use of barcodes, or 2D codes may provide a significantly more robust process for identifying different carriers than in existing multiplexed assay platforms, with minimum crosstalk between plex channels. In addition, the use of readable information in this way may enable the use of very much larger numbers of multiplex channels than is currently possible. For example, barcoding or 2D codes may enable 1000 plex, or 10,000 plex, or more if desired (i.e. an assay may involve 1000, or 10,000 or more multiplex channels). Use of barcodes or 2D codes may be particularly advantageous when each carrier is required to comprise unique readable information.

In a preferred embodiment, the (or each) carrier may comprise a magnetic material and a suitable external magnetic field may be applied to steer or move or drive the carrier through a fluid medium to a predetermined location for reading the code or information. The fluid medium may comprise or consist of the aqueous solution introduced to the carrier system to release the carrier. For example, a carrier having a high-aspect-ratio shape with a large top or bottom surface for displaying a code or information, may be directed so that it is in contact with a substrate or other supporting surface for convenient reading of the code or information. A magnetic field may be applied to steer the carrier to a reading position, and an assay result obtained by reading the readable code and the assay sample received on the carrier.

In a preferred embodiment, the carrier system may comprise a plurality of carriers for receiving an assay sample secured to the substrate by the release layer. An assay sample may be received on each of the carriers. The same type of assay sample may be received on each carrier. Alternatively, different types of assay sample may be received on some or each of the carriers. By providing a plurality of carriers, a multiplexed assay may advantageously be performed using the carrier system.

A second aspect of the invention may provide a method for manufacturing a carrier system for an assay, the method comprising the steps of: providing a substrate; forming a release layer on the substrate; and depositing or forming a carrier on the release layer such that the carrier is secured to the substrate, wherein the release layer is configured in use to release the carrier from the substrate in the presence of a biocompatible aqueous solution.

In a preferred embodiment, the step of forming the release layer on the substrate may comprise spin-coating the release layer. The release layer may comprise a material that is particularly suitable for spin-coating. For example, the release layer may comprise a sugar such as a dextran or a polyvinyl alcohol (PVA) or a poly(lactic-co-glycolic acid)) (PLGA) or a poly(acrylic acid) (PAA).

In a preferred embodiment, the step of depositing the carrier on the release layer may comprise fabricating the carrier on the release layer. In other words, the method of manufacturing the carrier system may comprise the step of fabricating the carrier. This conveniently may allow the carrier to be manufactured in the same process as the manufacture of the carrier system. Alternatively, the step of depositing the carrier may comprise depositing an already fabricated carrier on the release layer. For example, the carrier may have been manufactured by a third party.

A plurality of carriers may be deposited or fabricated on the release layer.

The method of manufacturing the carrier system may comprise a lithographic process. In particular, the carrier may be lithographically defined. This may advantageously provide a carrier having a high aspect ratio. The fabrication of the carrier may comprise a lithographic process. The lithographic process may comprise a physical vapour deposition process which advantageously enables sub-nanometer control in the deposition of the various layers used to fabricate the carriers. While the thickness of carriers manufactured using the lithographic process may be of the order of nanometers, the minimum size of the carriers in a lateral dimension may be between 5 and 200 microns, or between 20 and 200 microns; the carriers (in the lateral dimension) may be of any convenient shape, such as square or rectangular or circular. A carrier having a minimum lateral dimension of 5 or 10 or 20 microns may be suitable for receiving single cells and so advantageous in assays where single cells are of interest. A larger carrier, for example a carrier having a minimum lateral dimension of closer to 200 microns, may be suitable for receiving a plurality of cells. In a preferred embodiment, the carrier may have a minimum lateral dimension of 100 microns.

The fabrication of the carrier may comprise forming a photoresist layer on or over the release layer.

The photoresist layer may be spin coated over the release layer. As discussed above, the photoresist layer may advantageously comprise a material or materials that are not soluble in water, so that portions of the photoresist layer may be washed away, or dissolved, using a non-aqueous solvent that does not affect the release layer.

If the photoresist layer is not soluble in an aqueous solvent, then it may be considered biocompatible. For example, it may comprise a biocompatible polymer.

A suitable photoresist layer may be an SU-8 photoresist layer. An SU-8 photoresist layer may not be soluble in water. The lithographic process may comprise applying a photomask to the photoresist and exposing the photoresist layer to ultraviolet radiation, before washing away or dissolving unwanted regions of the photoresist layer.

Once the photoresist layer has been patterned to define the areas of the carriers on the substrate, the release layer in between the carriers may be removed, if desired. This may advantageously reduce the volume of the release layer which is exposed to the biocompatible solution during subsequent release of the carriers, reducing any concentration of the release layer that dissolves in the solution and minimising any influence that concentration may have on assay results.

During fabrication of carriers, subsequent layers of materials may be deposited on the carriers as described herein. These materials may also be deposited on the substrate or release layer between carriers. It is important that if any such materials are deposited between the carriers, they do not impede access of the biocompatible solution to the edges of the release layer beneath the carriers when an assay is performed. If required, materials between the carriers can be removed during fabrication to ensure that edges of the release layer beneath the carriers are exposed, so that the release layer can be activated by the biocompatible solution to release the carriers.

The method for manufacturing the carrier system may further comprise the step of sterilizing the carrier system by immersing it in ethanol, following the step of depositing or forming or fabricating the carrier on the release layer. Sterilization may advantageously eliminate unwanted forms of life or biological agents that might otherwise adversely interfere with assays performed using the carrier system. The carrier system may typically be immersed in the sterilization liquid for up to 10, 15, 20 or 25 minutes. Sterilization may comprise immersing the carrier system in a non-aqueous sterilization liquid such as ethanol. The sterilization liquid may comprise pure ethanol. This may be advantageous when the release layer comprises a material, such as dextran or polyvinyl alcohol, which is not activatable in a non-aqueous solvent, because the carrier will remain secured to the substrate while the carrier is immersed in the sterilization liquid. A similar effect may be achieved even if the sterilization liquid does affect the release layer, provided the release time of the release layer is greater than the period for which the carrier system is immersed in the sterilization liquid.

The step of sterilizing the carrier system may also be conducted using dry heat, steam, or gaseous sterilization methods. Dry heat sterilization may be the best suited for this as the release layer is least likely to be affected by this sterilization method.

The step of depositing or fabricating the carrier may comprise forming a magnetic structure or layer. In particular, when the method comprises a lithographic process, the method may comprise depositing a magnetic material on top of the photoresist layer. Additional materials such as gold may also be deposited on top of the photoresist layer or on top of the magnetic material of the carrier. A gold cap may provide biocompatibility. Furthermore, barcodes, QR codes or other readable information may be lithographically added to the carriers.

The method may further comprise the step of depositing a layer of gold on the carrier to form a cap. The gold cap may advantageously be biocompatible. The gold may be deposited only on a surface of the carrier that is exposed when the carrier is in contact with the substrate.

The method may further comprise adapting a surface of the carrier for receiving an assay sample. In a preferred embodiment, the surface of the carrier may be adapted for receiving adherent cells.

The step of adapting a surface of the carrier for receiving an assay sample may be performed before the step of depositing a carrier on the release layer. Alternatively, the step of adapting a surface of the carrier for receiving an assay sample may be performed after the step of depositing a carrier on the release layer. When the step of depositing the carrier on the release layer comprises fabricating the carrier on the release layer, the step of adapting the surface of the carrier for receiving the assay sample may be a step of that fabrication process.

The step of adapting a surface of the carrier for receiving an assay sample may comprise applying a coating to the surface of carrier, as has been described above.

The coating may be a charged coating. The coating may comprise a charged polymer. The charged polymer may be a polymer comprising a positive charge carrying group. The polymer may be a polyelectrolyte. The charged polymer may be a polymer comprising a positive charge carrying group. The polymer may be a polyornithine or a poly-d-lysine.

The polymer may be applied by immersing the carrier system in a solution comprising the polymer for a predetermined period of time, such as between 20 and 120 minutes. The solution may comprise the polymer in a concentration of between about 1 nM to 10 mM, depending on the polymer used. The solution may comprise a non-aqueous solvent such as ethanol. The solution may consist of the non-aqueous solvent and polymer. The use of a non-aqueous solvent may be advantageous when the release layer comprises a material, such as dextran or polyvinyl alcohol, that may not be activatable in a non-aqueous solvent. This may be because the carrier will remain secured to the substrate while the carrier is immersed in the solution comprising the polymer.

The polymer may comprise a thiol group and it may be the thiol group that covalently bonds with the gold cap of the carrier. This advantageously ensures that each polymer bonded onto the surface has the same orientation. Alternatively, the polymer may be adsorbed onto the gold cap of the carrier via Van der Waals forces.

Alternatively or additionally, the coating may comprise a plurality of ligands. The plurality of ligands may comprise antibodies. The plurality of ligands may comprise antibodies that specifically bind to cell receptors such as integrins. Alternatively or additionally the coating may comprise an extracellular matrix protein. The extracellular matrix protein may be a protein selected to increase or enhance cell adhesion. The protein may be a collagen, laminin or Matrigel, a protein mixture secreted by Engelbreth-Holm-Swarm (EHS) mouse sarcoma cells.

The step of adapting a surface of the carrier for receiving an assay sample may comprise modifying the surface topology of the carrier for example, by increasing the surface area of the carrier. The surface area may be increased by increasing roughness of the surface. For example, the surface may have been etched using an ion beam. Alternatively or additionally, dots, pits, protrusions or grooves may be provided on the carrier surface.

The step of adapting a surface of the carrier for receiving an assay sample may comprise modifying the hardness of a surface of the carrier. This may be achieved by the application of a coating comprising a dense layer of proteins or polymers.

A third aspect of the invention may provide a method of performing an assay using the carrier system as in the first aspect of the invention or manufactured as in the second aspect of the invention described above. The method comprises a step of introducing a biocompatible aqueous solution to the carrier system to release the carrier. The method may further comprise the step of introducing an assay sample to the carrier such that the assay sample is received by the carrier. The assay sample may advantageously be received by the carrier while the carrier is in contact with the substrate. An assay may be performed on a carrier having an assay sample received thereon. The advantages of the assay sample being received on the carrier while the carrier is in contact with the substrate have been described in relation to the first aspect.

In a preferred embodiment, the carrier may comprise a magnetic material and the method may further comprise applying a magnetic field to the carrier, the magnetic field acting to retain the carrier in contact with the substrate even after the release layer has released the carrier from the substrate. This may be particularly advantageous when the release layer releases the carrier in a time that is less than the typical time for an assay sample to be received by the carrier. For example, if the release layer comprises a sugar such as a dextran, the release layer, in the presence of a biocompatible aqueous solution, may release the carrier in five seconds or less. However, the typical time for an assay sample to be received by the carrier may be longer than five seconds. The assay sample may then advantageously be received by the carrier while the magnetic field retains the carrier in contact with the substrate (by magnetically applying a force urging the the carrier against the substrate) for the time required for the assay sample to be received by the carrier. This may remove the need to match the release time of the release layer with the typical time taken for a particular assay sample to be received by the carrier, while maintaining the advantages of receiving the assay sample on the carrier while the carrier is contact with the substrate.

The magnetic field may be applied to retain the carrier in contact with the substrate for at least five seconds. In other words, the magnetic field may be applied to retain the carrier in contact with the substrate for longer than the typical time for activating a release layer comprising a sugar such as a dextran in the presence of water. The magnetic field may be applied for much longer than five seconds. For example, the magnetic field may be applied for at least 1, 5 or 10 minutes, or at least 1, 3 or 12 hours. In a preferred embodiment, the magnetic field may be applied for 2 to 4 hours, or for about 3 hours, where the assay sample is an adherent cell. This is a typical time for an adherent cell to be received on the carrier. The magnetic field may typically be applied for less than 10 minutes, or less than 1, 3 or 12 hours, but this time will depend on the assay application.

It is clear that features described in relation to one aspect of the invention may be applied to other aspects of the invention.

A carrier system comprising a carrier or particle for receiving an assay sample has been described above. Particularly preferable features for the carrier have also been described above. However, it should be understood that the carrier system may comprise any carrier suitable for receiving and carrying a sample which may be secured to a substrate by a release layer and that such a carrier system may have the advantages described above.

Further specific preferred embodiments of the invention may include carriers on a wafer or substrate with or without pre-functionalization. A wafer may carry anywhere from 1000 -10 million carriers, or more, depending on customer needs. Alternatively, carriers on wafers or substrates may be provided with the wafers each in a well of a well plate. In this case there may typically be 100-10,000 carriers per wafer depending on the well size. An example of this embodiment may be a well plate containing the carriers and substrates already installed in one or more wells. In a further embodiment, carriers on a wafer or substrate may be provided with an assay sample, such as cells, in a frozen state already attached and ready to assay. Such a wafer may typically carry 1000-100,000 carriers.

Carrier systems embodying the invention may be provided to users in a variety of forms. In one embodiment, a carrier system comprising carriers on a substrate may be sterilised and packaged, for example in dry, sterile packaging or wrapping, for supply to users wishing to carry out assays. Such a carrier system may also be supplied in a sterile medium, which may be a liquid or gas. Alternatively, as described above carrier systems may be provided pre-loaded with assay samples, such as cells, usually frozen ready for use in an assay, In such carrier systems, the carriers may be suitably marked for identification, such as for identifying individual carriers, or groups of carriers.

PCT/GB2019/053188, a co-pending application filed by the applicant, describes the fabrication of lithographically defined carriers for multichannel assays. Many features described in PCT/GB2019/053188 are directly applicable to the present invention although in the context of the present invention the processes described in PCT/GB2019/053188 need to be modified to avoid using a water-soluble photoresist. For example, an SU-8 photoresist could be used. PCT/GB2019/053188 is incorporated herein in its entirety by reference, and is reproduced below in an Annex.

It may be noted that in the prior art, different types of magnetic particles for use in assays have been described. For example, patent publication WO2009/029859 describes a method for forming magnetic nanodiscs, of 1 nm to 200 nm width, by electrodeposition or chemical vapour deposition. The nanodiscs are formed either on a layer of sodium chloride or potassium chloride, which is then dissolved in water, or on a layer of copper, silver or aluminium, which is then dissolved in an acid/metal etchant. The product of this method is free nanodiscs in solution which are provided to users for use in DNA arrays. (The solution may be the solution which was used to remove the nanodiscs from the salt layer, or the nanodiscs may be washed and dispersed again into a suitable solution for use in DNA arrays.) The nanodiscs are formed with suitable molecules on their surfaces so that when they are in solution, they can bind to specific desired targets, i.e. particular structures on the surfaces of cells, so that those targets can be identified by use of a magnetic field sensor to sense the magnetic field of the nanodisc. The nanodiscs are too small to carry or support a cell, and are not usable in an assay on a substrate because they are much too small to support a cell. The nanodiscs may individually be influenced by an external magnetic field, but the volume of magnetic material in such a nanodisc is insufficient to direct an assay sample such as a cell within an assay, even after a nanodisc has bound to the assay sample.

Pat. Publication US2013/0052343 describes a another type of magnetic particle for targeting assay samples in solution. These particles are of 2 or 3 micrometres up to 100 micrometres or 500 micrometres diameter, and are formed by deposition in moulds defined in a layer of a photosensitive resin deposited over a layer of polymethyl methacrylate (PMMA). The photosensitive resin is selectively removed in the areas where particles are to be formed, the particles are deposited, and then the PMMA is dissolved in a solvent such as acetone to free the particles. The particles are then washed and suspended in a suitable solution for use. They are supplied in this form for users to target and enable recognition of desired molecular or cellular species.

### SPECIFIC DESCRIPTION

Specific embodiments of the invention will now be described by way of example, with reference to the accompanying drawings in which:

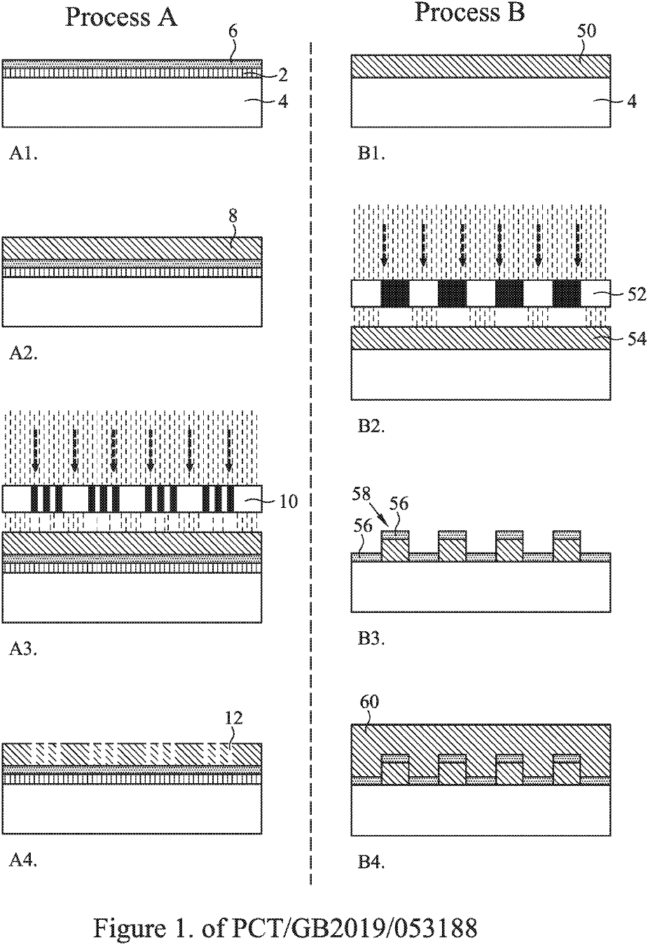

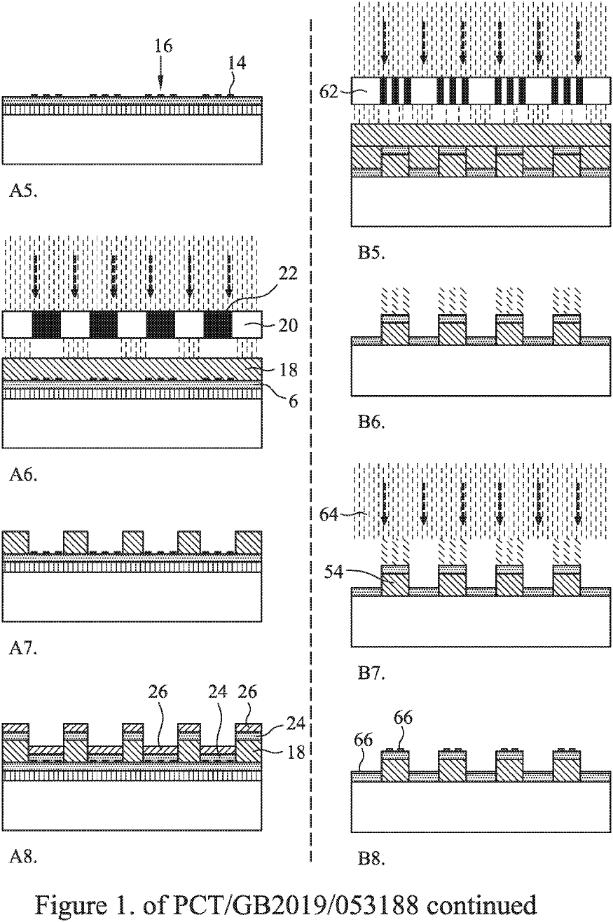

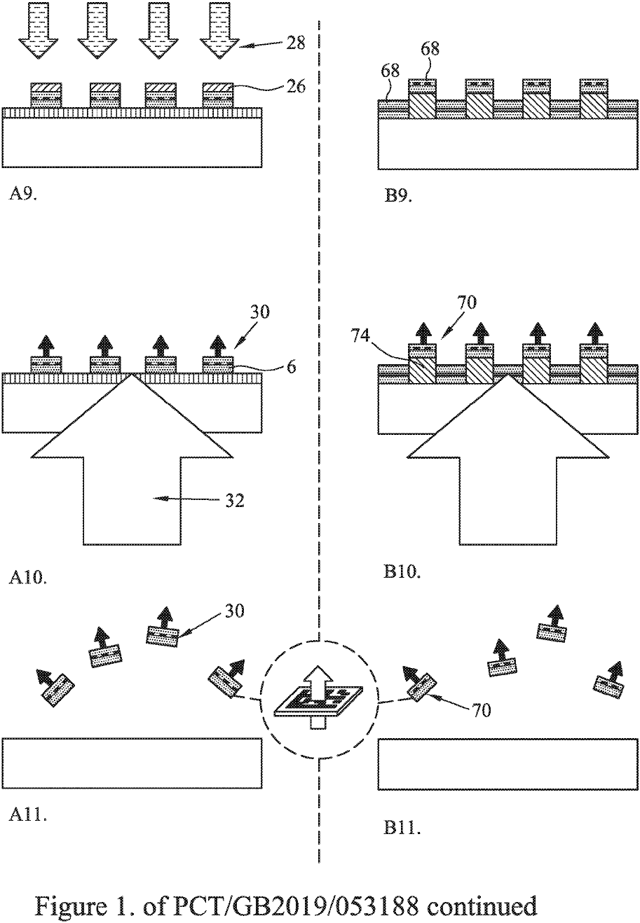

FIG. 1 is a schematic view of a carrier system comprising a plurality of carriers or particles secured to a substrate by a release layer, according to a first embodiment of the invention;

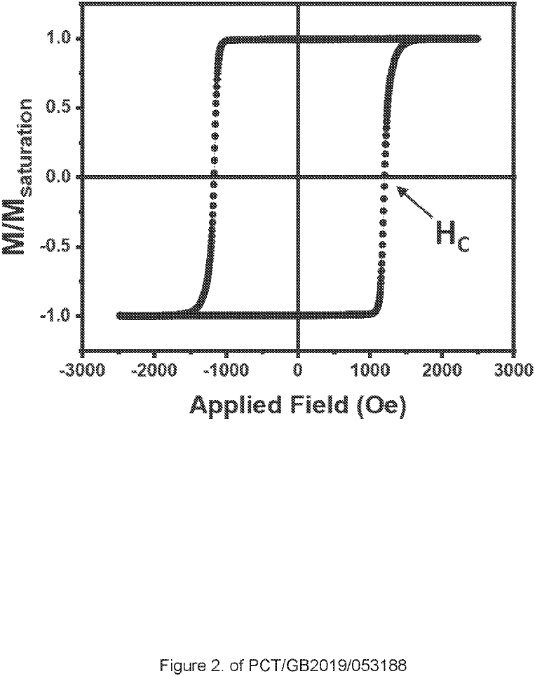

FIG. 2 illustrates a process for manufacturing the carrier system of FIG. 1;

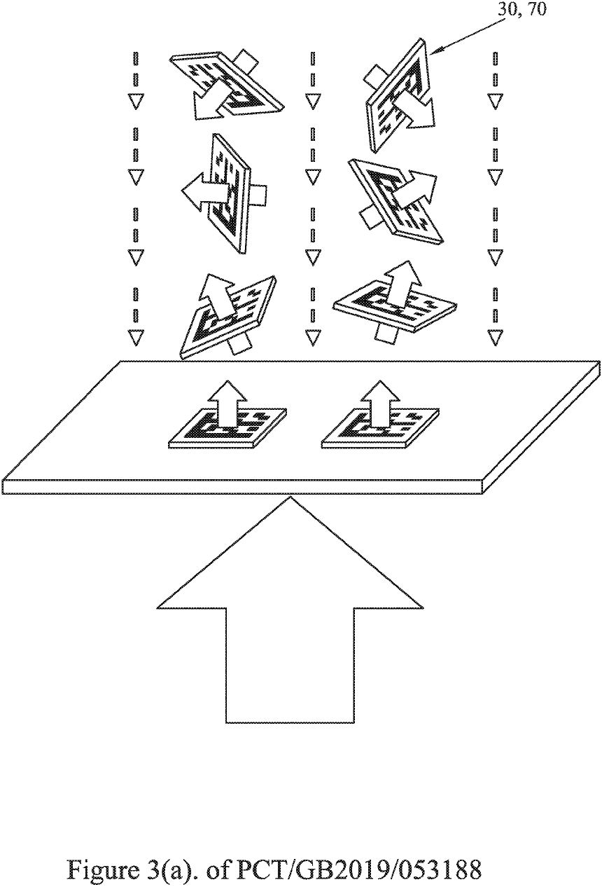

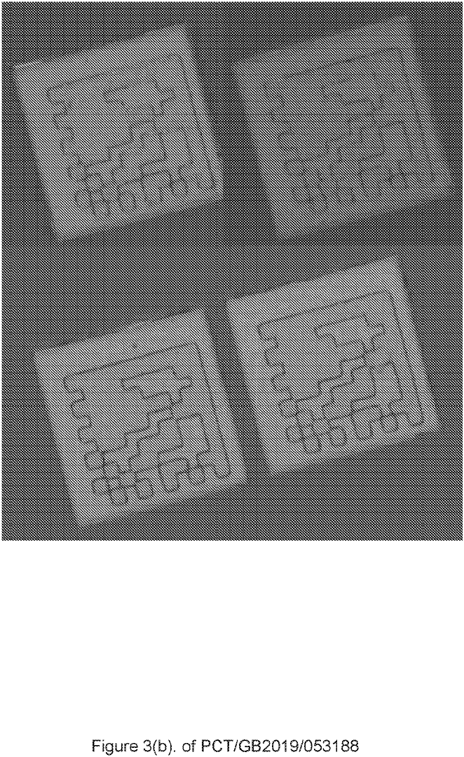

FIG. 3 shows a number of different surface adaptations of the carrier to improve the rate or efficiency with which adherent cells are received on the carrier; and

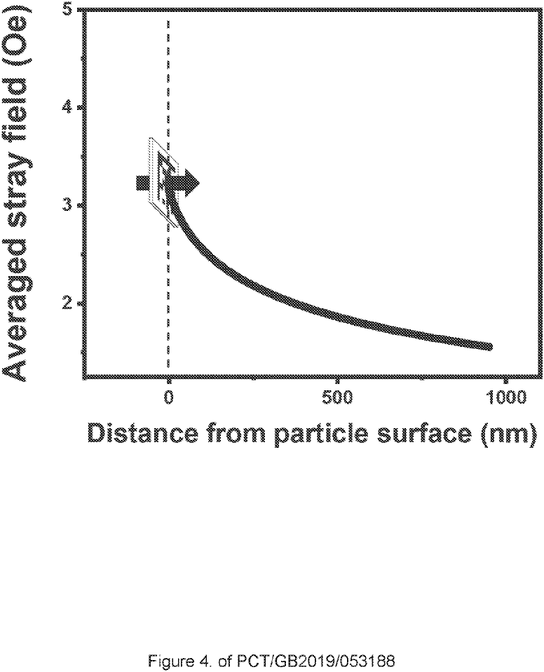

FIG. 4 shows a series of schematic views of one of the plurality of carriers of FIG. 1 during the process of introducing a biocompatible aqueous solution comprising an assay sample; FIG. 4ashows the carrier immediately prior to the introduction of the biocompatible aqueous solution comprising the assay sample, FIG. 4b shows a carrier that, following the introduction of the biocompatible aqueous solution, is no longer secured to the substrate by the release layer but that is retained in contact with the substrate by an external field, and FIG. 4c shows an assay sample received on a released carrier.

Sheets 5 to 13 of the accompanying drawings reproduce the drawings of co-pending application PCT/GB2019/053188 incorporated herein in its entirety by reference, and reproduced in the Annex below.

A carrier system 100 according to a first embodiment of the invention is shown in FIG. 1. The carrier system 100 comprises a substrate 10 in the form of a silicon chip. Four carriers, or particles, 12 are secured to the substrate 10 by a release layer 14 comprising a dextran. The carrier system can be used in an assay of adherent cells. Each of the carriers 12 is suitable for receiving an adherent cell or a plurality of adherent cells. Each carrier is in the shape of a flat, or high-aspect-ratio, cuboid with relatively large, square upper and lower surfaces. The upper surface 16 of each carrier, opposite to a lower side of the carrier contacting the release layer, is adapted for the cells to adhere to. The carriers have a lateral dimension of 100 microns. Each carrier comprises a layer of magnetic material, not shown in FIG. 1.

Each carrier comprises a barcode 18 such as a quick response (QR) code or 2D data matrix code. Predetermined readable codes are assigned to each carrier to identify the carriers. For example, the predetermined barcode 18 can refer to a particular adherent cell received on the carrier 12 or a particular adherent cell and particular reagent that the adherent cell is exposed to as part of an assay. In other words, the barcodes 18 allow for a multichannel assay to be performed by the carriers 12.

As shown in FIG. 1, the release layer 14 is discontinuous, so that the substrate is exposed 15 between each of the carriers 12. However, during manufacture the release layer 14 was originally formed by spin-coating a continuous layer of dextran covering the entire surface of the substrate 10, and the portions of the release layer between the carriers were removed after formation of the carriers. Therefore, the release layer 14 beneath the carriers may be considered a single layer.

While FIG. 1 shows four carriers 12 secured to the substrate 10, any number of carriers 12 can be secured to the substrate. A single carrier 12 can be secured to the substrate.

A lithographic process for manufacturing the carrier system 100 according to an embodiment of the invention is illustrated in FIG. 2.

FIG. 2a shows a release layer 14 formed on a silicon substrate 10 by spin-coating onto the substrate an aqueous solution comprising Dextran 70 in an amount 20 % by weight. Following the spin-coating of the release layer, the silicon substrate 10 is baked. In the case of a Dextran 70 release layer, the baking is at 150° C. for 2 minutes. A photoresist layer 20 is formed on top of the release layer 14 by spin-coating an SU-8 photoresist on top of the release layer 14. The SU-8 photoresist is then baked.

In an alternative embodiment, the release layer 14 can be formed of polyvinyl alcohol (PVA) rather than Dextran 70, by spin-coating an aqueous solution comprising a polyvinyl alcohol onto the substrate.

FIG. 2b shows a lithographic patterning step being performed on the SU-8 photoresist layer 20, by exposing the photoresist to ultraviolet radiation 24 through a patterned photomask 22 and baking the exposed resist at 95C for 2 minutes. This exposes specific regions of the photoresist layer 40 to the ultraviolet radiation 24 causing cross-linking in those regions. The remainder of the photoresist layer 24 remains soluble and can be washed away in a suitable non-aqueous solvent. This process allows for discontinuities to be formed in the photoresist layer to define the base of each carrier, without affecting the underlying release layer.

The barcodes are also added during the lithographic patterning of the SU-8 photoresist layer by making an array or pattern of holes in the SU-8. These holes can be read in transmission mode in a microscope and act as a barcode that can used to identify the carrier, or in the identification of the assay sample received on that carrier.

FIG. 2c shows the photoresist layer 20 following exposure to a suitable solvent. The primary solvents for SU-8 photoresists are PGMEA, gamma-butyrolactone or cyclopentanone. The soluble, non-cross-linked regions of the photoresist are washed away by the solvent to create a discontinuous SU-8 photoresist layer. Because gamma-butyrolactone or cyclopentanone are non-aqueous solvents, washing away the non-cross-linked regions of the photoresist layer does not dissolve the release layer 14. The release layer 14 is not soluble in non-aqueous solvents such as gamma-butyrolactone or cyclopentanone. Following washing, the remaining SU-8 forms the base 26 of each carrier.

An oxygen plasma etch is then applied, using the photoresist as a mask, to remove the release layer 14 from the substrate 10 in the regions between carriers. FIG. 2d shows the release layer and the overlying portions of the photoresist layer which form the bases 26 of the carriers, following this etching process.

FIG. 2e shows the carrier system after further layers 28 comprising magnetic materials have been deposited over the photoresist bases 26 of the carriers. These layers 28 include a plurality of layers of magnetic material interspersed with layers of non-magnetic material and are added by magnetron sputtering. In order to ensure that the carrier has a low stray field to avoid aggregation of the carrier, a layered structure comprising 11 layers is used, the layers being as follows (thickness in nm): Au(20.0)/Ta(2)/Pt(4)/CoFeB(0.6)/Pt(1.2)/CoFeB(0.6)/Pt(1.2)/CoFeB(0.6)/Pt(1.2)/ CoFeB(0.6)/Pt(5.0). It may be noted that the SU-8 photoresist layer of the carrier is about 1.5 to 2 microns in thickness, and advantageously provides mechanical stability to the carrier. Therefore, the magnetic layer is mechanically supported.

A cap of gold is then formed on the carrier using a top-down lithographic process. The gold cap is deposited on a surface of the carrier that is exposed while the carrier is secured to the substrate. The gold cap provides biocompatibility, and further coatings or surface adaptions can be applied to the gold cap depending on the desired assay application of the carriers.

FIG. 2f shows the carrier system after the gold cap layer has been adapted to be particularly suitable for receiving adherent cells by adding a coating 29 comprising a polymer.

The polymer comprises a thiol group and is applied by immersing the carrier system in a solution comprising the polymer for between 20 and 120 minutes. The solution comprises the polymer in a concentration of between about 10 µM to 10 mM. The polymer covalently bonds to the gold cap of the carrier by the thiol group. The solution comprises a non-aqueous solvent such as ethanol. The release layer is non-soluble in such a non-aqueous solvent and so the carrier will remain secured to the substrate while the carrier system is immersed in the solution comprising the polymer. FIG. 3a shows a schematic of a carrier 12 comprising a coating of charged polymers 32 comprising a thiol group covalently bonded to the carrier 12.

Alternative coatings can be applied to carrier. Instead of a charged polymer, the coating 29 can comprise a plurality of ligands comprising antibodies that specifically bind to cell receptors such as integrins or an extracellular matrix protein such as collagen, or Matigel, a protein mixture secreted by Engelbreth-Holm-Swarm (EHS) mouse sarcoma cells. FIG. 3b shows a schematic of a carrier 12 where a coating has been applied comprising a plurality of ligands 34.

Alternatively to applying a coating, the method can comprise modifying the physical surface of the carriers to be particularly suitable for receiving adherent cells, for example by modifying the gold cap. FIG. 3c shows an embodiment where the physical structure of the carrier, and particularly, the gold cap, has been adapted for receiving adherent cells. In this embodiment, the surface area has been increased by providing grooves 30 on the top surface of the carrier. The grooves 30 are formed by an extra lithography step or by etching the carrier (for example by plasma etching) during the manufacture process, before the deposition of the gold. The physical structure or topography of a surface can have an effect on the degree to which adherent cells adhere or seed onto that surface. Cell adhesion can be increased by increasing the surface area of the surface of the carrier.

The final step of the method of manufacturing the carrier system is sterilization, not shown in the figures. In order to sterilize the carrier system, it is immersed in pure ethanol for 20 minutes. This eliminates unwanted forms of life or biological agents that might otherwise adversely interfere with assays performed using the carrier system. Again, as dextran or polyvinyl alcohol are not soluble in such a non-aqueous solvent, the carrier remains secured to the substrate while the carrier system is sterilized.

FIG. 4 illustrates the carrier system in use in an assay of adherent cells 42, including a process of introducing a biocompatible aqueous solution 40 and adherent cells 42 to the carrier system 100. The adherent cells 42 to be received by the carrier 12 are transported in the biocompatible aqueous solution in order to maintain their viability. Once an adherent cell has been received by the carrier, the cell reaches a desired or natural adhered morphology. The coating 29 on the carrier, described above, improves the efficiency or rate at which the cells are received by carrier and so reduces the time required for the cells to be received on the carrier and increases the strength of bonding or attraction between the assay sample and the carrier. The assay sample is thus advantageously securely bonded to the carrier.

In practice, the carrier system 100 would be placed in a container and immersed in the biocompatible aqueous solution. This is schematically represented in FIG. 4a by the test tube 44 containing a quantity of biocompatible aqueous solution comprising the adherent cells 42 and the drop of biocompatible aqueous solution 40. The biocompatible aqueous solution 40 and the adherent cells 42 are not drawn to scale.

When the biocompatible aqueous solution 40 comes into contact with the release layer 14 comprising dextran, the release layer 14 dissolves. This releases the carrier 12. Because dextran is biocompatible and non-toxic, dissolving the dextran release layer 14 in the aqueous solution 40 has a minimal effect on the adherent cells 42 and so a minimal effect on the results of the assay to be performed on the adherent cells 42. FIG. 4b shows the release layer 14 as completely dissolving to release the carrier 12. However, in other embodiments, using different materials for the release layer, the release layer 14 may not dissolve or may not dissolve completely. In such cases, it is only required that the bond between the release layer 14 and the carrier 12 is weakened such that the carrier 12 is released.

In the presence of a biocompatible aqueous solution, a dextran release layer 14 will typically release the carrier in five seconds or less. This is considerably shorter than the typical time taken for the adherent cell to be received by the carrier. It may be necessary to allow three hours or more for the adherent cell to be received by the carrier 12. However, it is preferred for the adherent cell to be received on the carrier while the carrier is in contact with the substrate. This ensures that the outer, or top, surface 16 of the carrier 12 remains the main exposed surface of the carrier, preventing adherent cells 42 from being received, for example, on a bottom surface of the carrier 12.

As illustrated in FIG. 4b, in order to retain the carrier in contact with the substrate even after the release layer has released the carrier, an external magnetic field is applied. As described above, the carriers 12 comprise layers 28 that include magnetic material. The external field is arranged to apply a force to the carrier 12 that urges the carrier against the substrate and holds it in contact with the substrate 10 even after the release layer 14 has released the carrier 12. The external field is represented by the dotted arrows in FIG. 4b.

In an alternative embodiment, the release layer can be designed with a release time that matches or exceeds the typical time for the adherent cell to be received on the carrier. In that case, the step of applying an external field, as shown in FIG. 4b, may not be necessary. A release layer comprising a polyvinyl alcohol is biocompatible and can be made to have a longer release time, such as twelve hours or longer. This can advantageously be achieved through a combination of formulation of the PVA layer (hydrolysis degree in the range 85-99%, or a ratio of two PVAs with different hydrolysis degrees) and baking, typically at about 115C. If the baking step is omitted, PVA layers with much shorter release times, down to a few minutes or less, can be fabricated. In this way the release time of the release layer can be designed as required for specific assay applications.

Therefore, when a carrier system 100 comprising a suitably-fabricated polyvinyl alcohol release layer 14 is used with adherent cells that typically take three hours to be received by the carrier 12, there may be no need to apply an external field to retain the carrier in contact with the substrate, as described above.

Once the carrier has been released from substrate, the released carrier 12 is free to move through the aqueous solution 20 surrounding the carrier system. This is shown in FIG. 4c. External magnetic fields can be used to apply forces to the carrier in order to manipulate and move the carrier as desired to perform the assay. The biocompatible aqueous solution 20 also provides a suitable environment for the adherent cells 22 to maintain their viability. It is more convenient to manipulate and transport adherent cells received on carriers than trying to manipulate and transport the adherent cells on their own, particularly because adherent cells are most viable when received on a surface. Receiving the cells on the carriers 12 allows each adherent cell to be transported as if it is in suspension while remaining in a biologically representative, adhered state.

Once the carriers having adherent cells received thereon are released from the substrate, an assay can be performed.

By way of a summary, the following numbered clauses set out various preferred embodiments of the invention:

1. A carrier system for an assay comprising a particle or carrier secured to a substrate by a release layer, the particle or carrier being suitable for receiving an assay sample, and the release layer being configured in use to release the particle or carrier from the substrate in the presence of a biocompatible aqueous solution.

2. A carrier system as in clause 1, wherein the release layer is configured such that, following activation of the release layer, the biocompatible aqueous solution remains biocompatible.

3. A carrier system as in clause 1 or 2, wherein the release layer comprises a material that is water-activatable, such as a material that is water-soluble.

4. A carrier system as in any preceding clause, wherein the release layer is not activatable in a non-aqueous solvent such as ethanol.

5. A carrier system as in any preceding clause, wherein the release layer comprises at least one of a sugar, such as a dextran, or a polyvinyl alcohol.

6. A carrier system as in any preceding clause, wherein the particle comprises a magnetic material;
wherein the particle preferably comprises a layered structure between a top surface of the particle and an opposed bottom surface of the particle, the layers including one or more magnetized layers;
in which the ratio of a lateral dimension of the one or more magnetized layers to a thickness or aggregate thickness of the magnetized layer or layers is particularly preferably greater than 500.

7. A carrier system as in any preceding clause, wherein the particle is lithographically defined.

8. A carrier system as in any preceding clause, wherein the particle comprises a photoresist layer, such as an SU-8 photoresist.

9. A carrier system as in any preceding clause, wherein a surface of the particle is adapted for receiving the assay sample, for example wherein the surface comprises a gold cap layer on to which a polymer is covalently bonded by thiol group.

10. A carrier system as in any preceding clause, wherein the particle comprises readable information, such as a readable code selected from a barcode or a 2D code.

11. A carrier system as in any preceding clause, comprising a plurality of particles, wherein each of the particles is secured to the substrate by the release layer.

12. A method of manufacturing a carrier system for an assay, the method comprising the steps of:
providing a substrate;
forming a release layer on the substrate; and
depositing a particle or carrier for receiving an assay sample on the release layer such that the particle or carrier is secured to the substrate,
wherein the release layer is configured in use to release the particle from the substrate in the presence of a biocompatible aqueous solution.

13. A method as in clause 12 wherein the step of forming the release layer on the substrate comprises spin-coating the release layer.

14. A method as in clause 12 or 13, wherein the step of depositing the particle on the release layer comprises fabricating the particle on the release layer, for example by a lithographic process.

15. A method as in any of clauses 12 to 14, wherein the release layer is adapted to release the particle from the substrate, in use, in the presence of the biocompatible aqueous solution within a time of between 1 hour to 72 hours.

16. A method as in any of clauses 12 to 15, further comprising the step of adapting a surface of the particle such that the surface is suitable for receiving an assay sample.

17. A method as in any of clauses 12 to 16, further comprising the step of sterilizing the carrier system by immersing the carrier system in ethanol, following the step of depositing the particle on the release layer.

18. A method as in any of clauses 12 to 17, wherein the step of depositing a particle comprises forming a magnetic structure.

19. A method of performing an assay using the carrier system as defined in any of clauses 1 to 11, the method comprising the step of:

introducing a biocompatible aqueous solution to the carrier system to release the particle or carrier.

20. A method as in clause 19, further comprising the step of introducing a sample for an assay to the particle such that the sample is received by the particle.

21. A method as in clause 20, wherein the sample is received by the particle while the particle is in contact with the substrate.

22. A method of performing an assay as in clause 21, wherein the particle comprises a magnetic material and the method further comprises applying a magnetic field to the particle, the magnetic field acting to retain the particle in contact with the substrate even after the release layer has released the particle from the substrate.

23. A method of performing an assay as in clause 22, wherein the magnetic field is applied to retain the particle in contact with the substrate for at least five seconds, or at least one minute, or at least 5 minutes or at least 30 minutes.

24. A method of using the carrier system as defined in any of clauses 1 to 11, comprising the steps of introducing a sample for an assay to a particle or carrier such that the sample is received by the particle while the particle is in contact with the substrate, and storing the sample received on the particle or carrier, preferably by freezing the sample received on the particle.

25. A method as defined in clause 24, further comprising the step of releasing the particle from the substrate before storing the sample received on the particle.

### ANNEX

For reference, the contents of international patent application PCT/GB2019/053188, a co-pending application filed by the applicant, are reproduced below. This includes a detailed description of the fabrication of a magnetic carrier, or particle, similar to that in an embodiment of the present invention, differing in that in the present invention the carrier is fabricated or attached to a release layer. If the carrier is fabricated on the release layer, then lithographic processes which do not prematurely activate the release layer must be used instead of those described in the Annex. One such option is to replace the photoresist described in the Annex with a photoresist which can be washed away without using an aqueous solvent. The figures of PCT/GB2019/053188 are set out at sheets 5/13 to 13/13 of the drawings.

### PCT/GB2019/053188: Magnetic Carrier and Method

The invention relates to a magnetic carrier, and methods of making and using magnetic carriers.

### PCT/GB2019/053188: Background Art

Techniques for using an applied magnetic field to exert mechanical forces on individual magnetic carriers have been leveraged in various biotechnology applications.

One important commercial use for magnetic nanocarriers and microcarriers is currently for bioassays, to isolate and identify biological molecules. Superparamagnetic iron-oxide nanocarriers (SPIONs) are conventionally used for this commercial range of applications, because they offer the property of migrating towards an external magnetic field source. This enables carriers to be steered by the application of an external magnetic field towards a desired location for reading the assay information. These carriers are nanocarriers (5-20 nm in diameter) made by colloidal chemistry methods. The size of these carriers is limited by the fact that if the carriers are made larger, beyond approximately 20 nm, the carriers become ferromagnetic and the effect of the stray magnetic field of one carrier on other carriers disadvantageously leads to magnetic agglomeration of the carriers, which prevents their use in bioassays.

Technologists developing magnetic carriers need to optimize the magnetic properties of the carriers for each application. Conventionally, it is accepted that a highly-desirable property for magnetic nanocarriers for all of these various applications, including the bioassay applications, is a net-zero magnetization remanent state in order to avoid carrier agglomeration.

A net-zero-magnetization remanent state means that in the absence of a magnetic field, the magnetic carriers have no net magnetic moment and no external stray field. In use, magnetic carriers are typically suspended in a liquid or fluid medium and are free to move within that medium. For carriers with non-zero remanent moments, the carriers’ stray fields may interact and cause the carriers to agglomerate, or to clump together. This is undesirable because the purpose of using magnetic carriers in biotechnology applications is to be able to steer, or direct the motion of, carriers suspended in a liquid or fluid medium by applying an external magnetic field. If the magnetic carriers agglomerate, then this cannot be achieved.

In addition, it is understood in the art that to ensure that small magnetic fields from the environment cannot cause agglomeration by inducing magnetic moments in magnetic carriers, the carriers with zero net magnetization remanent state should also have a low susceptibility at small fields.

Furthermore, if carriers with a high susceptibility are used, then after an applied field has been applied to direct or move the carriers in a desired manner, then carriers which have agglomerated during the application of the field stay agglomerated once the applied field is removed. It is understood by the skilled person that this should also be avoided in magnetic carriers for biotechnology applications.

Thus the prior work in the art of creating magnetic nanocarriers that do not agglomerate has focussed entirely on systems that have a zero net remanent magnetization state, preferably with low magnetic susceptibility. This includes a variety of systems such as superparamagnetic nanocarriers, magnetic vortex micro and nanocarriers, and micro and nanocarriers utilizing antiferromagnetic coupling to create opposing magnetization configurations between adjacent magnetic layers in the carrier.

An important biotechnology application is to carry out multiplexed immunoassays of biological samples. The accurate quantification of proteins in a biological sample is of significant importance for both research and clinical diagnostic applications. A multiplexed immunoassay simultaneously quantifies a plurality of different proteins in a given sample. Analysing protein fingerprints of samples in this way has the potential to accelerate research and to enable improved diagnostics. In response to this market need, multiplexed assay systems such as Luminex (RTM), Firefly (RTM) and Fireplex (RTM) have been developed. These systems use individual carrier sets in which each carrier is coated with a capture antibody qualified for one specific analyte. Multiple sets of analyte-specific carriers can then be combined to detect and quantify multiple targets simultaneously through the use of detection antibodies marked with fluorescent labels. The Luminex (RTM) system is based on polystyrene or paramagnetic microspheres, or beads, that are internally dyed with red and infrared fluorophores of differing intensities to allow the differentiation of one set of beads from another. The Firefly (RTM) and Fireplex (RTM) systems also use fluorophores to allow differentiation of one carrier set from another, but in this case the carriers are in the form of rods coded by applying a different fluorophore at each end. Measurement of the fluorophores again aims to distinguish one rod from another. However, in practice these systems suffer from limited multiplexing (limited number of different proteins that can be identified) due to limited ability to distinguish with certainty between the channels of the multiplex in assay results.

### PCT/GB2019/053188: Summary of Invention

The invention provides a magnetic carrier, a plurality of magnetic carriers for performing assays, and a method for performing an assay using the magnetic carrier(s), as defined in the appended independent claims to which reference should now be made. Preferred or advantageous features of the invention are set out in dependent subclaims.

In a first aspect the invention may thus provide a magnetic carrier, a layered structure between a top surface of the carrier and an opposed bottom surface of the carrier, the layer or layers including one or more magnetized layers. The ratio of a lateral dimension of the one or more magnetized layers to the thickness, or aggregate or effective thickness, of the magnetized layer or layers is greater than 500. In other words, the aspect ratio of a cross section of the magnetized layer or layers may be more than 500. In preferred embodiments, the ratio may be higher, for example being greater than 800 or greater than 1000 or 1500 or 2000.

The carrier may further comprise a non-magnetic layer, which may advantageously provide mechanical support to the magnetic layer, and may determine physical characteristics of the carrier such as its mechanical properties and its density.

The carrier may comprise one magnetized layer, or it may comprise more than one such layer. If it comprises two or more magnetized layers, then layers may be adjacent to each other, or in contact with each other, or they may be spaced from each other with non-magnetic material in between. The aggregate thickness, or total thickness, of the magnetically-remanent layers in a carrier having more than one magnetized layer may be the sum of the thicknesses of those magnetic layers, not including any non-magnetic layers in between them. In some embodiments, the layered structure may comprise many magnetized layers and/or many layers of a non-magnetic material.

A magnetized, or magnetic, layer may for example comprise any suitable magnetic material or materials, such as a ferromagnetic material, element or alloy, or a composite of superparamagnetic nanocarriers.

The carrier is preferably substantially flat in shape, comprising one or more substantially flat layers of magnetic and/or non-magnetic materials stacked on top of one another. The layers are preferably of substantially the same shape and size as each other, each having the same lateral shape and size as the carrier itself. However, the shape and structure of the carrier may vary from this as described further below.

The carrier may have a zero or a non-zero magnetic remanence. However, the inventors have found that even if a carrier has non-zero magnetic remanence, then the shape and structure of a carrier embodying the invention displays an unexpectedly-low stray field at the surfaces of the carrier, so that a plurality of the carriers suspended in a fluid or liquid medium may advantageously not aggregate or clump. Surprisingly, the inventors have found that this is the case whether or not the carriers are fully magnetically remanent.

In a preferred embodiment of the invention, when an external field is applied the carrier has a sufficient magnetic moment for the external field to apply a desired force to the carrier. The desired force may depend on the application of the carrier (such as in a bioassay with the carrier suspended in a fluid medium). In practical implementations, the applied external field is typically less than 2 T, or 1 T or 0.5 T, and may typically be greater than 0.05 T or 0.1 T or 0.25 T.

For example, in a bio- or chemical assay, it may be desired to use the external field to steer the carrier within a fluid medium. The magnetic moment may be due to magnetization of the carrier itself, or it may be induced in the carrier by the external field. It is important, however, for the carrier to contain sufficient magnetic material to enable the desired force to be generated.

The magnetic moment that can be generated by an external field applied to a carrier may depend on the total volume V of magnetic material in the carrier multiplied by that material’s magnetization Mₛ. Thus, the value of V.Mₛ for a carrier embodying the invention is preferably greater than a predetermined value, such as 10⁻¹⁸ J/T or 5×10⁻¹⁸ J/T or 10⁻¹⁷ J/T.

The inventors have found that the physical distribution of the magnetic material within the carrier may determine the stray field near the carrier, and therefore the tendency of carriers to interact with each other and/or to agglomerate. The inventors have found that distributing the magnetic material in the form of a layer or layers (preferably parallel layers) having a cross section with high aspect ratio AR may generate an advantageously low stray field. This preferred carrier geometry may advantageously provide carriers with low stray field and little or no tendency to agglomerate.

To evaluate this carrier geometry in a more quantitative manner, the inventors suggest that in preferred carriers, the parameter AR/Mₛ² of the magnetic layer or layers (with AR being a dimensionless ratio and Mₛ being the magnetization of the magnetic material in the magnetic layer or layers measured in A/m (1000 A/m is equivalent to 1 emu/cm3)) is preferably greater than 8×10⁻¹⁰ m⁻²/A⁻², or 1.2×10⁻⁹ m⁻²/A⁻² or 8×10⁻⁹ m⁻²/A⁻².

Alternatively, or in addition, the inventors have determined that the parameter AR/Mₛ is preferably greater than 1×10⁻³ m⁻¹/A⁻¹ or 3×10⁻³ m⁻¹/A⁻¹ or 5×10⁻³ m⁻¹/A⁻¹.

These limits correspond to a stray field of less than about 2500 A/m (30Oe) at 10 times the layer thickness above or below the layer. Depending on the application and environment of the carriers, this level of stray field may advantageously prevent agglomeration.

When assessing the value of AR for a carrier structure, AR may be a lateral dimension of a cross section of the structure divided by a thickness of the structure. For a layer of magnetic material, the lateral dimension may be the minimum lateral dimension of the layer or, if the shape of the layer is more complex, then it may be preferable to consider an average lateral dimension of the layer. If the thickness of the layer is constant, then that thickness can be used for the calculation of AR. If the thickness of the layer varies, then an average thickness can be used.

It may be appropriate to use equivalent lateral and thickness dimensions of the carrier itself to calculate AR, for example if the magnetic material spans the full lateral dimension of the carrier, and particularly if the thickness of the carrier is sufficiently similar to the thickness of the magnetized layer or layers, such as less than 10 times, or 5 times, the thickness of the magnetized layer or layers. In this approach to evaluating AR/Ms, as well as using the AR value for the carrier, the value of Mₛ may be modified by calculating a diluted Mₛ value, being the Mₛ of the magnetized material in the carrier multiplied by the volume ratio of non-magnetized and magnetized material in the carrier.

A carrier may comprise multiple magnetized layers, for example in the form of a stack of magnetized layers spaced by layers of non-magnetized material. In such cases, if the magnetized layers are spaced from each other by a sufficiently small distance, such as less than 5, 10 or 20 times the thinnest layer thickness or the average layer thickness, then AR may be evaluated using either the aggregate thickness of the magnetized layers or the distance between the outermost magnetized layers in the stack, including the thickness of any intervening non-magnetic layers.

If a carrier comprises multiple layers that span a sufficient proportion of the carrier thickness, then AR may be evaluated using the carrier thickness.

If a carrier comprises multiple layers, an alternative approach to evaluating the thickness for calculating AR may be to calculate a diluted thickness for the magnetized material. If for example, two or more parallel layers of magnetized material of aggregate thickness Tm are separated by layers of non-magnetic material of aggregate thickness Tnm, then the diluted thickness of the magnetized material would be Tm/(Tm+Tnm) .

It may also be possible to evaluate AR and Ms (for the calculation of AR/Mₛ² or AR/Mₛ) for an entire carrier by measurement. If the magnetic moment of a set number of carriers of unknown structure but known dimensions is measured (for example using a vibrating-sample magnetometer), the effective Mₛ may be found using the total carrier volume and the total moment per carrier. The carrier or metallic layer(s) lateral dimension and thickness may be measured directly, for example using microscopy and/or electron microscopy techniques, to evaluate AR.

Carriers embodying the invention are preferably planar in shape, with their length and width both being greater than their thickness. Advantageously the length and width of the carrier, or two lateral dimensions of the carrier measured perpendicular to each other, are similar to each other, or differ from each other by less than about 10%, 30%, 50% or 70%. A carrier might typically be in the form of a circular or elliptical or polygonal disc, or a generally flat cuboid with a square or rectangular perimeter.

The magnetic material in a carrier preferably extends across substantially the entire lateral dimensions of the carrier, in the form of a layer or layers within the carrier. The aspect ratio of the magnetic layer or layers can be assessed with reference to the minimum lateral dimension, or an average lateral dimension, of the layer or layers, which may be the same as or smaller than the lateral dimension of the carrier. If one magnetized layer is present, then the aspect ratio AR may be the minimum, or an average, lateral dimension of the magnetic layer divided by its thickness (or an average thickness if the thickness varies). If multiple magnetized layers are present, then the aspect ratio AR may be evaluated as the lateral dimension, or an average lateral dimension if different layers have different lateral dimensions, divided by an aggregate thickness of the layers.

In preferred embodiments, the top and bottom surfaces of the carrier may be separated by a carrier thickness of between 5 nm, or 10 nm or 50 nm or 100 nm, and 100 µm or 50 µm or 5 µm or 1 µm or 500 nm. A minimum lateral dimension of the carrier may be greater than 1 µm, and preferably greater than 5 µm or 10 µm, and a maximum lateral dimension may be less than 500 µm or 200 µm or 100 µm or 50 µm. A ratio of the minimum lateral dimension of the carrier to the thickness of the carrier may be greater than 10 or 20 or 50 and/or less than 2000 or 1000 or 500. In such preferred embodiments, the carrier may therefore have a rather flat, high-aspect ratio shape, although other embodiments envisage lower-aspect-ratio carrier shapes, and even spherical or cube-shaped carriers. Such a lower-aspect-ratio carrier may comprise one or more magnetized layer(s) having a higher aspect ratio, as discussed above and herein.

In embodiments comprising more than one magnetized layer, those layers are preferably substantially parallel to each other. In embodiments comprising more than one magnetized layer, those layers preferably have similar shapes and/or areas as each other, and may conveniently overlap with each other, optionally completely overlapping with each other.

The opposed top and bottom surfaces of the carrier are advantageously flat, but one or both surfaces may optionally be curved or not flat without affecting the desired property of the carrier having a sufficiently small stray field to avoid aggregation. The carrier itself may thus be flat or curved. But in each case, the opposed top and bottom surfaces may advantageously be of sufficiently large area to enable features such as readable information, such as readable codes in the form of barcodes or 2D codes, to be applied to the top and/or bottom surfaces so that individual carriers or groups of carriers (if the carriers in a group of carriers are similarly marked) can be identified by reading the information. Such information may be applied to the top and/or bottom surfaces, or may be applied beneath the top and/or bottom surfaces, for example beneath a surface layer or layers that is or are sufficiently transparent to allow the codes or information to be read through the surface layer or layers. Further, the top or bottom surfaces may advantageously form suitable substrates for the application of other functionality to the carriers, such as biofunctionality or chemical functionality for biotechnological or chemical applications as described herein.

In a preferred embodiment, the shape of the carrier may thus be in the form of a thin (low thickness) laterally-extended shape, such as a high-aspect-ratio cuboid or disc. (Aspect ratio refers to the ratio of the minimum lateral dimension, or of an average lateral dimension, to the thickness.) Alternatively, the magnetic carrier may be described as being cylindrical in shape, the thickness of the carrier being in an axial direction of the cylinder, with the peripheral shape of the cylinder preferably being selected so that it typically has an edge or edges which are convex or straight, advantageously with no re-entrant corners. Preferred peripheral shapes are rectangular or square or circular.

While being of a high-aspect-ratio or cylindrical shape, as described above, it is contemplated that while the carrier is preferably flat, with flat top and bottom surfaces, embodiments of the invention may include curved or non-flat carriers, or carriers with curved or non-flat upper and lower surfaces, while achieving the object of providing non-aggregating magnetic carriers.

Preferably, the minimum and maximum lateral dimensions of the magnetic carrier differ by less than 90% or less than 70%. In preferred embodiments, the minimum lateral dimension of the carrier is greater than 5 µm, and preferably greater than 10 µm, and/or the maximum lateral dimension of the carrier is less than 500 µm, and preferably less than 200 µm, 100 µm or 75 µm. These dimensions may be selected by the skilled person depending on the application for which the carriers are being used, and requirements such as the desired mechanical strength of the carriers.

The layered structure of the magnetic carrier advantageously comprises a magnetized layer and a non-magnetic layer. The non-magnetic layer may provide mechanical strength to the carrier, and may provide a suitable substrate for the magnetized layer. The non-magnetic layer may thus advantageously comprise a material selected from Al, Ta, Pt, Pd, Ru, Au, Cu, W, MgO, Cr, Ti, Si, Ir, SiO₂, SiO, Sn, Ag, polymers, plastics, alloys of these materials, and composites or mixtures comprising these materials.

The carrier may comprise two or more layers of non-magnetic material, which may similarly be selected from this group.

The magnetized, or ferromagnetic, layer may be formed from any suitable material, and in preferred embodiments may comprise a material selected for example from metals or metallic alloys such as Fe, Co, Ni, CoFe, CoFeB, FePt, CoNi and NiFe.

The magnetized layer may for example comprise a magnetic multilayer stack of alternating layers of a magnetic material and a noble metal (such as Pt/CoFeB) where the pair is known to provide perpendicular magnetic anisotropy.

The magnetized, layer is preferably an out-of-plane magnetized layer, but may be a differently magnetized layer such as an in-plane magnetized layer.

A high saturated magnetic moment is desirable for the magnetic carriers, in order to achieve a rapid response to an external field. The magnetic material is selected to achieve this.

The layered structure of the magnetic carrier may comprise more than one layer of non-magnetic material, and/or may contain more than one layer of magnetized material. In a preferred embodiment, the carrier may comprise a magnetized layer positioned between two layers of non-magnetic material.

A carrier may comprise two or more magnetized layers arranged, in combination, to have zero magnetic remanence in the absence of an applied field. Such a carrier may have a magnetic susceptibility such that the application of an external field induces a magnetic moment in the carrier. An external field may therefore be applied in order to move or steer the carrier, for example through a fluid medium. Advantageously, however, it may not matter whether the carrier has a high or low susceptibility because the shape of the carrier embodying the invention is such that the stray field around the carrier may advantageously be too low to cause carrier agglomeration even when the magnetic moment is induced.

In such a carrier, the magnetized layer may advantageously be spaced from the top surface of the carrier by more than 25% of the carrier thickness, and spaced from the bottom surface of the carrier by more than 25% of the carrier thickness. This structure may advantageously further reduce stray magnetic fields at the opposed top and bottom surfaces of the carrier.

Preferably, the magnetized layer may have a thickness, or an average thickness, greater than 0.1 nm, or 0.4, 1.0 or 1.5 nm. Preferably, the thickness of the magnetized layer may be less than 25%, and particularly preferably less than 15% or 10%, of the total thickness of the carrier. The carrier could comprise only a magnetized layer, if the mechanical strength of the carrier were sufficient for a desired application.

The magnetized layer may for example be a thin-film multilayer.

The net magnetic field (the stray field) averaged across the lateral surface at or within a small distance of a top or bottom surface of the carrier may preferably be less than 2500 A/m (30Oe)and particularly preferably less than 800 A/m (10Oe) or 400 A/m (5Oe). This field may be measured at the surface, or at a small distance such as 10 nm, 50 nm or 100 nm from the surface, for example by using a magnetic atomic force microscope. The inventors’ experiments have indicated that these external, or stray, magnetic fields are sufficiently small to avoid agglomeration of magnetic carriers.

Magnetic carriers embodying the invention may conveniently be manufactured or fabricated by lithographic processes.

A second aspect of the invention may advantageously provide a magnetic carrier having dimensions as described above, but preferably in which a top surface of the carrier and an opposed bottom surface of the carrier are separated by a carrier thickness of between 5 nm and 200 µm, a minimum lateral dimension of the carrier is greater than 1 µm, and the ratio of the minimum lateral dimension to the thickness is greater than 10, and in which the carrier comprises a layered structure through its thickness, the layers including one or more magnetically-remanent, or magnetized, layer(s) and one or more layer(s) of a non-magnetic material. Such a carrier may conveniently be fabricated by a lithographic process, and may comprise one or more of the features of the first aspect of the invention described herein.

In a further aspect of the invention, a top or bottom surface of the carrier may carry readable information, such as a readable code. This may be for example a barcode or 2D code. This may allow the carrier to be identified remotely by reading the information, for example with a camera and suitable software.

In a preferred embodiment, the magnetic properties of the carrier enable a suitable external magnetic field to be applied to steer or move or drive the carrier through a fluid medium to a predetermined location for reading the code or information. For example, a carrier having a high-aspect-ratio shape with a large top or bottom surface on which a code or information is carried, may be directed so that it is in contact with a substrate or other supporting surface for convenient reading of the code or information.

In a still further aspect of the invention, a top and/or bottom surface of the carrier may be functionalised, for example biofunctionalized or chemically functionalised. This may advantageously be in combination with applying readable information to the carrier. For example, a top or bottom surface of the carrier may carry a readable code and the same or an opposite surface may be functionalised. Further, in a preferred embodiment, a plurality of carriers may be provided in which each carrier carries readable information corresponding to the functionalisation of that carrier.

Such a carrier may enable the performance of an assay, such as a bioassay, by providing the carrier to a liquid or fluid assay sample and allowing the functionality of the carrier to interact with the assay sample, for example with biological molecules or other components of the assay sample. A magnetic field may be applied to steer the carrier to a reading position, and an assay result obtained by reading the readable code and measuring the interaction of the carrier’s functionality with the assay sample.

A multi-channel assay may be performed by providing a plurality of carriers in which each carrier carries readable information corresponding to the different functionality of that carrier. The plurality of carriers may contain groups of carriers, the carriers in each group carrying similar readable information and being similarly functionalised. The plurality of carriers can be contacted with a liquid or fluid assay sample, allowing the functionality of the carriers to interact with the assay sample. A magnetic field is applied to steer the carriers to a reading position, and an assay result obtained by reading the readable information for two or more carriers and measuring the respective interaction of the corresponding functionalities of each carrier with the assay sample.

The identification of carriers using readable information in this way may advantageously provide a multiplexed platform in which the carriers can be accurately distinguished from each other. The use of barcodes, or 2D codes, for example may provide a significantly more robust process for identifying different carriers than in existing multiplexed assay platforms, with minimum crosstalk between plex channels. In addition, the use of readable information in this way may enable the use of very much larger numbers of multiplex channels than is currently possible. For example, barcoding or 2D codes may enable 1000 plex, or 10,000 plex, or more if desired.

In preferred embodiments, the invention may thus relate to lithographically defined, perpendicularly (or out-of-plane) magnetized carriers, advantageously in the form of ferromagnetic microdiscs (microcarriers, nanocarriers, microcarriers etc.) for use in biotechnology applications. For example, these carriers may be ferromagnetic microcarriers or microdiscs (between 1- 500 µm, or 1- 200 µm, or preferably 5 - 100 µm, in each lateral dimension, or in two orthogonal lateral dimensions, and between 10 nm and 200 µm, or preferably 20 nm and 10 µm thick) fabricated by photolithography and by the physical vapour deposition of magnetized thin film multilayers. For example, the carriers may be circular or square, 40 µm in diameter or side length, and 100 nm thick. Or they may be 100 µm in diameter or side length, and 1 µm thick. The resultant high planar aspect ratio, ultra-thin discs, or microdiscs, (which may be referred to as magnetic carriers (MCs) because of their ability to carry functionalisations such as biofunctional antibodies for diagnostic tools) are ferromagnetic with high magnetic moment. The MCs may be lithographically-defined. The MCs do not agglomerate when suspended in a fluid because the aspect ratio of each magnetic layer (typically 1 nm, or 5 nm, total magnetic layer thickness and tens of µm in lateral size), and magnetization direction perpendicular to the plane of the MC results in a negligible stray magnetic field from each carrier.

In preferred forms, the MCs may be characterized by a magnetization direction parallel to the surface normal of the microdisc, as well as coercive magnetization reversal, and a high magnetic anisotropy. These properties may all enable a higher degree of control over their magnetic response and hence their mechanical behaviour in a fluid under the influence of an external magnetic field.

The physical vapour deposition process which is preferably used to fabricate the carriers, or MCs, enables sub-nm control in the deposition of the magnetic thin films that form the MCs, and thus offers extreme precision in the engineering in the magnetic properties of the MCs. This may advantageously enable them to be tailored to different applications. Further, barcodes (or other readable information) may be lithographically added to the surface of MCs, and the surface materials may be chosen for optimal functionalization with molecules of interest.

### PCT/GB2019/053188: Specific Embodiments and Best Mode of the Invention

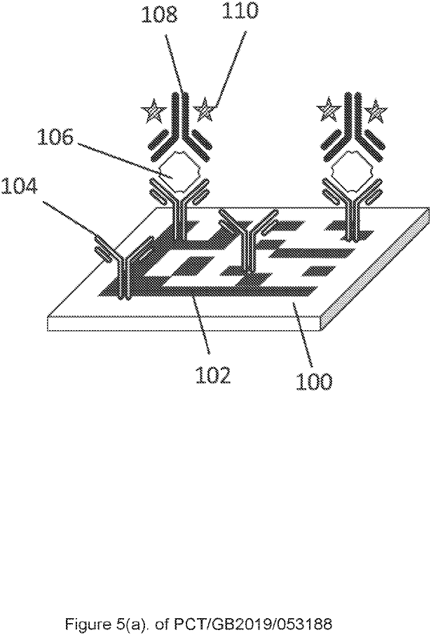

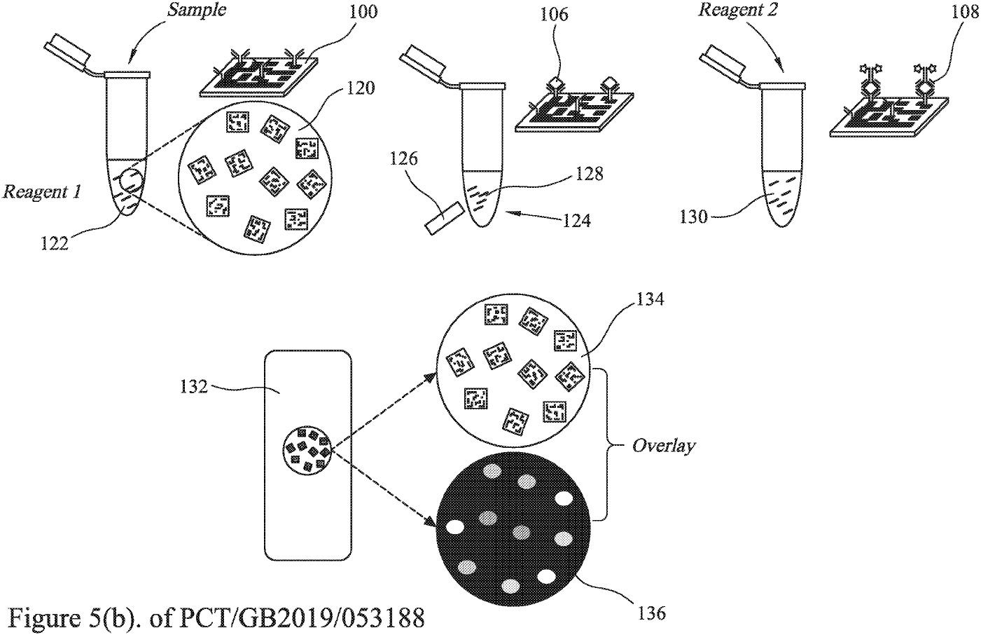

Specific embodiments of the invention will now be described by way of example, with reference to the accompanying drawings in which:
FIG. 1 illustrates steps in two processes, Process A and Process B, for the fabrication of magnetic carriers according to first and second embodiments of the invention;
FIG. 2 is a polar magneto-optical Kerr effect (MOKE) measurement of the magnetic response of the magnetic thin film Au(100.0)/Ta(2)/Pt(4)/CoFeB(0.6)/Pt(1.2)/ CoFeB(0.6)/Pt(1.2)/CoFeB(0.6)/Pt(1.2)/CoFeB(0.6)/Pt(5.0) used in the carriers of the embodiments;
FIGS. 3(a) and 3(b) illustrate how the readable code and the magnetic states in carriers according to the embodiments are linked to ensure that the readable code may always be aligned to an external detector, such as a camera or barcode reader, by an applied magnetic field, and show images of readable codes of carriers imaged by a detector;
FIG. 4 illustrates stray field strength as a function of distance from the surface of a carrier manufactured according to the embodiments; and
FIGS. 5(a) and 5(b) illustrate a functionalised carrier according to a further embodiment of the invention, suitable for a bioassay, and illustrate the use of carriers according to the further embodiment of the invention to implement a streamlined multiplex assay.

A specific embodiment of the invention involves the fabrication of high magnetic moment microcarriers made from ultrathin perpendicularly-magnetized CoFeB/Pt layers. The high aspect ratio of the shape of these carriers results in an extremely low stray magnetic field from each carrier, such that the magnetic nanocarriers show no inter-carrier interaction (and therefore no agglomeration). When an external magnetic field is applied, the carriers transition to magnetic saturation with coercive, sharp switching and are fully remanent. Individual barcodes are added to the carriers using a simple and robust lithography process and can be read optically. As described below, a robust multiplexed assay, for example a cytokine assay, using the magnetic carriers has been demonstrated highlighting their potential in assay applications.

In the embodiment, lithographically fabricated magnetic carriers may advantageously achieve high magnetic moment, no intercarrier interaction, a large surface area for functionalization, and robust carrier specific barcoding. These carriers may be referred to as magnetic carriers (MCs) in view of their ability to carry both functionalization and readable information. The large surface area of the carriers may advantageously provide more area for functionalisation than in conventional assay carriers.

Lithographically defined magnetic nanocarriers are known in the prior art, for example in T. Vemulkar, R. Mansell, D. C. M. C. Petit, R. P. Cowburn, and M. S. Lesniak, “Highly tunable perpendicularly magnetized synthetic antiferromagnets for biotechnology applications,” Appl. Phys. Lett., 2015, in H. Joisten et al., “Self-polarization phenomenon and control of dispersion of synthetic antiferromagnetic nanocarriers for biological applications,” Appl. Phys. Lett., vol. 97, no. 25, p. 253112, 2010, and in S. Leulmi et al., “Comparison of dispersion and actuation properties of vortex and synthetic antiferromagnetic carriers for biotechnological applications,” Appl. Phys. Lett., vol. 103, no. 13, p. 132412, 2013. But in stark contrast to these lithographically defined carriers and other magnetic nanocarriers in general, the MCs used here do not require the engineering of a net zero remanent magnetization state to prevent carrier agglomeration. The MCs used here may optionally have net-zero remanence (and susceptibility to the generation of a magnetic moment in an external field) but despite the conventional expectation of the skilled person, they do not require net-zero remanence to avoid agglomeration. The stray field of the carriers is sufficiently low to avoid agglomeration due to the shape of the magnetized material in the carrier, and/or the shape of the carrier, whether or not the remanent magnetization in the absence of an external field is zero.

The MCs in the embodiment are extremely high aspect ratio cuboids, with planar length and width of 40 microns, and thickness of approximately 150 nanometres.

Two lithographic processes (A and B) according to two embodiments of the invention for the fabrication of magnetic carriers, or MCs, are illustrated in FIG. 1.

Process A is illustrated in FIG. 1.A1 to 1.A11. In FIG. 1.A1 a sacrificial layer 2 of 50 nm of Al is grown by magnetron sputtering on a Si substrate 4. The base 6 of the carrier thin-film stack is then grown on top of this sacrificial layer, also by magnetron sputtering. This base consists of the following 11 layers (thickness in nm):

Au(100.0)/Ta/Pt(4)/CoFeB(0.6)/Pt(1.2)/CoFeB(0.6)/Pt(1.2)/CoFeB(0.6)/Pt(1.2)/ CoFeB(0.6)/Pt(5.0).

In FIG. 1.A2 a photoresist 8 is spin-coated over the MC base 6. The photoresist is then exposed during lithographic patterning using a photomask 10 defining the carrier barcodes (or readable codes) as in FIG. 1.A3. This standard photolithography process creates a plurality of holes 12 in the photoresist illustrated in FIG. 1.A4, in which a barcode contrast material 14 such as 15 nm of Ta is deposited on top of the carrier base using magnetron sputtering as shown in FIG. 1.A5. The shape and pattern of the holes defines the barcodes 16.

The photoresist is then removed in a solvent such as acetone, and a new layer of photoresist 18 is spin-coated on top of the carrier base 6 and the barcodes 16 as shown in FIG. 1.A6. This is exposed in a second lithographic patterning process using a mask 20 to define the shape of the carriers. In this step shown in FIG. 1.A7, the plurality of holes 22 that define the carrier shape are aligned such that the barcodes are aligned at the centres of the holes.

In FIG. 1.A9 a carrier (MC) cap 24, and an ion-beam-milling hard mask 26 are added by magnetron sputtering, which consist of 30-40 nm Au, and 200 nm Al respectively. The thickness of gold is selected to ensure complete coating of the carriers (on both top and bottom surfaces) with Au for biocompatibility and to provide a surface for biofunctionalization. The thickness of the Au is however sufficiently thin to allow the barcode to be read through the Au layer.

In FIG. 1.A9 the photoresist 18 is then removed in a solvent such as acetone, and then the entire sample is subjected to ion-beam milling 28, a standard subtractive patterning process. Any thin film not protected by the ion-beam-milling hard mask is milled away. Thus, the milling removes all of the thin film that forms the base of the carrier thin film stack that is not within the defined carrier shapes. The milling process is stopped when the sacrificial layer is reached. Any remaining Al hard mask 26 may be removed by dissolution in a 10-30 min soak in 3-5% tetramethylammonium hydroxide solution, or equivalent Al solution etchant.

Thus, photolithography patterning determines the planar shape of the carriers, and the physical vapour deposition process determines their thickness and composition.

At this stage the carriers 30 with barcodes, the MCs, are fully defined and lie on top of the sacrificial layer. A magnetic field 32 greater than the coercive field for the magnetic thin film of the carriers is then applied to ensure that all of the carriers are magnetised out-of-plane, in an “up” state, perpendicular to the top and bottom surfaces of the carriers, as shown in FIG. 1.A10. Alternatively, the carriers may all be magnetised in a “down” state. This links the magnetization of the carriers to the physical structure of the carriers in the vertical direction, allowing for alignment of the barcodes in any downstream steps such as re-deposition as shown in FIG. 3 or analysis in solution.

Finally, as shown in FIG. 1.A11, the sacrificial layer of Al beneath the carriers is dissolved in an appropriate solvent to lift the carriers 30 off the substrate and release them into solution in a fluid medium.

Process B is illustrated in FIG. 1.B1 to 1.B11. In FIG. 1.B1 a photoresist layer 50 is spin-coated over a Si substrate 4. It is then exposed in FIG. 1.B2 using a photomask 52 to create a plurality of islands or pillars of photoresist 54, on which a series of layers of material 56 are deposited in FIG. 1.B3 using magnetron sputtering to form the base 58 of the layered, thin-film structure of the magnetic carriers. The shape of the islands or pillars defines the shape of the carriers. FIG. 1.B3 thus illustrates the structure after the deposition of the first layers of the carrier. These, listed from the bottom up, consist of the following thin film layers (thicknesses in nm): Au(100.0)/Ta(2)/Pt(4)/CoFeB(0.6)/Pt(1.2)/ CoFeB(0.6)/Pt(1.2)/CoFeB(0.6)/Pt(1.2)/CoFeB(0.6)/Pt(5.0).

A lithographically-defined barcode is then added to the carriers. As shown in FIG. 1.B4 a second layer of photoresist 60 is applied, and is exposed as shown in FIG. 1.B5 using a photomask 62 patterned with the desired barcode for each carrier In FIG. 1.B6-7 the photoresist is developed, and then flood-exposed 64 to allow for its removal in developer downstream. The bottom layer of each photoresist island or pillar 54 is shielded from this exposure step by the presence of the carriers on top of the islands.

A barcode contrast material 66 such as 15 nm of Ta is grown on top of the carriers in FIG. 1.B8. The top layer of photoresist is then completely removed using developer in FIG. 1.B9 and the carrier cap 68 is deposited consisting of 30-40 nm of Au. The bottom layer of resist remains intact. The thickness of gold is selected to ensure complete coating of the MCs (on both top and bottom surfaces) with Au for biocompatibility and to provide a surface for biofunctionalization. The thickness of the Au is however sufficiently thin to allow the barcode to be read through the Au layer.

Thus, photolithography patterning determines the planar shape of the carriers, and the physical vapour deposition process determines their thickness and composition.

At this stage, the carriers with barcodes, the MCs, 70 are fully defined and lie on top of the islands of photoresist. A magnetic field 72 greater than the coercive field for the magnetic thin film of the carriers is then applied to ensure that all of the carriers are magnetised out-of-plane, in an “up” state, perpendicular to the top and bottom surfaces of the carriers, as shown in FIG. 1.B10. Alternatively, the carriers may all be magnetised in a “down” state. This links the magnetization of the carriers to the physical structure of the carriers in the vertical direction, allowing for alignment of the barcodes in any downstream steps such as re-deposition as shown in FIG. 3 or analysis in solution.

The thin film structure of the MCs described in this embodiment in Processes A and B is thus defined as a base of (thicknesses in nm): Au(100.0)/Ta(2)/Pt(4)/CoFeB(0.6)/Pt(1.2)/ CoFeB(0.6)/Pt(1.2)/CoFeB(0.6)/Pt(1.2)/ CoFeB(0.6)/Pt(5.0). A 15 nm Ta barcode is on top of this layer, and this is then capped with 30-40 nm of Au. The thinner Au at the top face allows for imaging the barcode through the Au, and thus the barcode is only visible through the top face of the carrier in the embodiment described here. Thus linking the carrier magnetization to the physical structure of the carrier at this juncture is necessary to enable control and orientation of the barcoded face of the carrier once in solution.

Finally, as shown in FIG. 1.B11, the photoresist 74 beneath the carriers is dissolved in an appropriate solvent to lift the carriers 70 off the substrate and release them into solution in a fluid medium.

FIG. 2 is a polar magneto-optical Kerr effect (MOKE) measurement of the magnetic response of the magnetic thin film Au(100.0)/Ta(2)/Pt(4)/CoFeB(0.6)/Pt(1.2)/ CoFeB(0.6)/Pt(1.2)/CoFeB(0.6)/Pt(1.2)/CoFeB(0.6)/Pt(5.0) used in the carriers of the embodiments. The thin film magnetization is clearly out of plane with sharp, coercive magnetic switches to saturation. H_{c} denotes the coercive field, or the field required to magnetically switch the thin film to its saturated magnetic state.

FIG. 3a shows how the barcode (or other 2D code) and the magnetic states of the carriers 30, 70 are linked to ensure that the code may always be aligned to an external detector. First, as described above, a field above H_{c} is used to set the magnetization of the carriers in the “up” state before the carriers are lifted off into solution. Once in solution, the carriers will retain this magnetization state if they are not exposed to any strong magnetic field pulses. Any applied field less than H_{c} will simply result in the carriers rotating to align their magnetic moment M with the externally applied field. Field strengths from 10 - 1000 Oe, and frequencies from 0-50 Hz, may typically be used to control the movement of the carriers. Since the barcodes are fabricated on the top surface of each carrier and coated with 30-40 nm of Au through which they can be optically imaged, aligning the magnetic moment M with an external magnetic field corresponds to uniquely aligning the barcoded face of the carriers in the direction of the applied magnetic (H) field. As shown in FIG. 3, the carriers may thus be directed by an applied magnetic field onto a substrate, such as a planar substrate, for reading by a barcode reader and any associated detector, such as a fluorescence detector or camera.

FIG. 3b shows images of carriers on a planar substrate and displaying readable codes such as barcodes.

FIG. 4 illustrates the stray field strength as a function of distance from the surface of a carrier 30, 70 of the embodiments. It can be seen that the stray field is low, due to the high-aspect-ratio geometry of the carrier. This advantageously reduces any tendency for the carriers to agglomerate.

Carriers according to an embodiment of the invention may be used to implement a multiplex assay as follows. Steps in the process are illustrated in FIGS. 5 (a) and (b). Each carrier 100 has been lithographically fabricated, for example as described above, and patterned with a barcode 102 (preferably a quick response (QR) code or 2D data matrix code. Predetermined barcodes, or other readable codes, are assigned to desired assay samples, such as particular proteins to be identified in a multichannel bioassay. Each carrier carrying a code corresponding to a particular protein is then functionalised with capturing antibodies 104 specific to that respective protein as shown in FIG. 5a. This can be performed using conventional biochemistry protocols. The gold surfaces of the carriers are suitable for this functionalisation.

In this and other embodiments, if other functionalisation of the carriers is required, then materials other than gold may be used for one or both of the top and bottom surfaces of the carrier. For example, SiO₂ may be used.

Analyte detection is performed with a conventional sandwich immunoassay. When the capturing antibody captures a target protein 106, exposure of the magnetic carrier to a fluorescently-labelled detection antibody 108 complementary to the capturing antibody binds to and labels the protein. As the skilled person understands, fluorescence of the fluorophore 110 in the detection antibody can then be used to indicate that the protein has been captured, and was therefore present in the sample tested in the assay.

A convenient multiplexed analyte capture platform can thus be prepared for any desired application, comprising a plurality of sets (or groups) of magnetic carriers, each set of carriers carrying a unique code and functionalised with the corresponding capture antibody. For a desired range of target proteins, the plurality of sets of carriers corresponding to those target proteins can be mixed together in an assay sample, such as a patient sample on which a diagnosis is to be performed using a multichannel assay.

In an assay according to a preferred embodiment, illustrated in FIG. 5b, an analyte reagent consists of a desired set 120 of functionalised magnetic carriers carried in a fluid medium 122. In a typical example, the analyte reagent may include a set or group of approximately 100-1000 coded magnetic carriers (MCs) functionalised with the capturing antibody for each target protein 106. The analyte reagent is mixed with the sample to be analysed, and is allowed to react with any target proteins present. The magnetic carriers are then removed from the sample and the fluid medium by magnetic separation 124. This involves attracting the carriers together using an external magnetic field 126 (for example so that they gather 128 at the bottom of a container holding the sample) and the sample removed or decanted. The carriers are then re-suspended in a fluid medium 130 and exposed to the corresponding fluorescently-labelled detection antibodies 108. Through the application of an external magnetic field the carriers are then driven, or steered, and positioned on a surface 132 for reading. The surface may be a glass slide for example. Notably, because each carrier has been magnetised out-of-plane, with the magnetisation in a unique direction towards or away from the top surface of the carrier, the carriers can be steered so that they are all in the same plane as each other, on the surface for reading, and so that they are all oriented in the same way, for example with the top surface of each carrier facing away from the reading surface.

In an alternative embodiment, the carriers may be magnetised in-plane, with the magnetic field parallel to the top or bottom surface of the carriers. The carriers can then be directed by the external magnetic field onto the surface for reading, but they cannot all be aligned with the top or bottom surface of each carrier facing away from the surface. This apparent problem may be solved in one of two ways. The carriers may be fabricated with readable information on both the top and bottom sides of the carriers, so that the information may be read from either side. Alternatively, the carriers may be fabricated such that the readable information can be read from either side of the carrier, for example by making the carrier layers sufficiently transparent that the information can be read from both the top and bottom surfaces.

Once the carriers have been positioned on the surface 132 for reading, two images of the carriers can be taken using suitable cameras and control software. A first image 134 is a bright field image showing the codes or information on each carrier. This unambiguously identifies which of the carriers in the image is carrying the capture antibody for each target protein, or in other words to which channel of the multichannel assay each carrier belongs. A second image 136 is a fluorescence image of the carriers. If a carrier fluoresces, then the detection antibodies on that carrier have captured the corresponding protein, and the fluorescence intensity may indicate the concentration of the protein in the sample. If a carrier does not fluoresce, then that carrier has not captured its corresponding protein, which is therefore not present in the sample. An overlay of the two images can therefore identify which proteins were present in the sample by assigning a fluorescence intensity value to each carrier. Corresponding analysis software may then indicate which proteins are present in the sample, and the concentrations of those proteins.

A significant feature of the multichannel analysis enabled by the barcoding of the carriers is that the potential number of plex channels is extremely large, up to as many channels as can be coded by the barcodes, which may even be 1000 channels or more. At the same time, the carriers in individual channels can be unambiguously identified, achieving little or no crosstalk between the channels. By comparison, conventional bead based bioassays use fluorescence-based channel identification systems which are much less resistant to crosstalk. For example, one prior art system uses ratios of fluorophores for barcoding beads, and a fluorophore-labelled antibody as the positive signalling for analyte detection. This creates challenges in reliability of channel identification and severely limits plex numbers.

### PCT/GB2019/053188: Clauses Setting Out Preferred Features

1. A magnetic carrier, comprising a layered structure between a top surface of the carrier and an opposed bottom surface of the carrier, the layers including one or more magnetized layers;

in which the ratio of a lateral dimension of the one or more magnetized layers to a thickness or aggregate thickness of the magnetized layer or layers is greater than 500.

2. A magnetic carrier according to clause 1, in which the layers include a non-magnetic layer.

3. A magnetic carrier according to clause 1 or 2, in which the ratio of the lateral dimension of the one or more magnetized layers to the thickness or aggregate thickness of the magnetized layer or layers is greater than 1000, and preferably greater than 2000.

4. A magnetic carrier according to clause 1, 2 or 3, in which the magnetized layer or layers comprise a volume V of magnetic material having a magnetisation or average magnetisation Ms, a cross section of the layer or layers has an aspect ratio AR, and AR/Ms² (with Ms measured in A/m) is greater than 8*10⁻¹⁰(A/m)⁻².

5. A magnetic carrier according to any preceding clause, in which the magnetized layer or layers comprise a volume V of magnetic material having a magnetisation or average magnetisation Ms, a cross section of the layer or layers has an aspect ratio AR, and in which AR/Ms (with Ms measured in A/m) is greater than 0.001 (A/m).

6. A magnetic carrier according to any preceding clause, in which the top and bottom surfaces of the carrier are separated by a carrier thickness of between 5 nm and 200 µm, and/or a minimum lateral dimension of the carrier is greater than 1 µm, and preferably greater than 5 µm or 10 µm.

7. A magnetic carrier according to any preceding clause, in which a ratio of the minimum lateral dimension of the carrier to the thickness of the carrier is greater than 10.

8. A magnetic carrier according to any preceding clause, in which a maximum lateral dimension of the carrier is less than 1000 µm, and preferably less than 500 µm or 200 µm,.

9. A magnetic carrier according to clause 8, in which the minimum lateral dimension of the carrier is at least 10% of the maximum lateral dimension of the carrier, and preferably at least 30% or 50% or 70% of the maximum lateral dimension.

10. A magnetic carrier according to any preceding clause, in which a lateral periphery of the carrier is of a shape comprising convex or straight sides, and is preferably of a shape having no convex sides and/or re-entrant corners.

11. A magnetic carrier according to any preceding clause, in which the lateral dimensions of the magnetic layer or at least one of the magnetic layers are the same as the lateral dimensions of the carrier.

12. A magnetic carrier according to any preceding clause, in which the non-magnetic layer comprises a material selected from non-magnetic metals, non-metals, semi-metals and compounds, Al, Ta, Pt, Pd, Ru, Au, Cu, W, MgO, Cr, Ti, Si, Ir, SiO2, SiO, Sn, Ag, SiN, Ge, polymers, plastics, alloys of these materials, and composites or mixtures thereof.

13. A magnetic carrier according to any preceding clause, in which the magnetized layer or each of the magnetized layers comprises a material selected from magnetic metals, magnetic alloys, magnetic compounds and superparamagnetic nanocarrier composites, such as Fe, Co, Ni, CoFe, CoFeB, FePt, CoNi, NiFe and Fe₂O₃.

14. A magnetic carrier according to any preceding clause, in which the magnetized layer or each of the magnetized layers is an out-of-plane-magnetized layer.

15. A magnetic carrier according to any of clauses 1 to 13, in which the magnetized layer or each of the magnetized layers is an in-plane-magnetized layer.

16. A magnetic carrier according to any preceding clause, in which the magnetized layer or each of the magnetized layers is positioned between the layer of a non-magnetic material and a second layer of a non-magnetic material.

17. A magnetic carrier according to clause 16, in which the magnetized layer or each of the magnetized layers is spaced from the top surface of the carrier by more than 25% of the carrier thickness, and is spaced from the bottom surface of the carrier by more than 25% of the carrier thickness.

18. A magnetic carrier according to any preceding clause, in which the magnetized layer or each of the magnetized layers has a thickness greater than 0.1 nm, and preferably greater than 0.5 nm.

19. A magnetic carrier according to any preceding clause, in which the aggregate thickness of the magnetized layer or layers is less than 25%, and preferably less than 15% or 10%, of the thickness of the carrier.

20. A magnetic carrier according to any preceding clause, in which the magnetized layer or each of the magnetized layers is a thin-film multilayer.

21. A magnetic carrier according to any preceding clause, in which the net magnetic field (the stray field) averaged across the top or bottom surface of the carrier is less than 2500 A/m and preferably less than 800 A/m or 400 A/m.

22. A magnetic carrier according to any preceding clause, fabricated by lithography.

23. A magnetic carrier according to any preceding clause, in which the carrier carries readable information, such as a readable code selected from a barcode or 2D code, which is readable at or from one or both of the top or bottom surface of the carrier.

24. A magnetic carrier according to clause 23, in which a surface of the carrier is functionalised, and in which the readable information corresponds to the functionality of that carrier.

25. A magnetic carrier as defined in clause 24, in which a top or a bottom surface of the carrier is functionalised and in which the information is readable at or from the same surface as is functionalised;

and/or in which a surface of each carrier is functionalised and in which each carrier carries readable information corresponding to the functionalisation of that carrier.

26. A method for making a magnetic carrier as defined in any of clauses 1 to 25, by a lithographic process.

27. A method for performing an assay, comprising providing a carrier as defined in clause 24 or 25 to a liquid assay sample, allowing the functionality of the carrier to interact with the assay sample, applying a magnetic field to steer the carrier to a reading position, and obtaining an assay result by reading the readable information and the interaction of the functionality of the carrier with the assay sample.

28. A method for performing a multi-channel assay, comprising providing a plurality of carriers as defined in clause 24 or 25 to an assay sample, allowing the functionality of the carriers to interact with the assay sample, applying a magnetic field to steer the carriers to a reading position, and obtaining an assay result by reading the readable information for two or more carriers and the interaction of the corresponding functionalities of those carriers with the assay sample.

1. A carrier system for an assay comprising a carrier secured to a substrate by a release layer, the carrier being suitable for receiving an assay sample, and the release layer being configured in use to release the carrier from the substrate in the presence of a biocompatible aqueous solution.

2. A carrier system according to claim 1, adapted to receive the assay sample onto the carrier while the carrier is in contact with the substrate.

3. A carrier system according to claim 1 or 2, in which the assay sample is suspended in the biocompatible aqueous solution, and in which when the carrier system is contacted with the biocompatible solution, the assay sample is received onto the carrier and the carrier is released from the substrate.

4. A carrier system according to any preceding claim, wherein the release layer is configured such that, following activation of the release layer, the biocompatible aqueous solution remains biocompatible.

5. A carrier system according to any preceding claim, wherein the release layer comprises a material that is water-activatable, such as a material that is water-soluble.

6. A carrier system according to any preceding claim, wherein the release layer is not activatable in a non-aqueous solvent such as ethanol.

7. A carrier system according to any preceding claim, wherein the release layer comprises at least one of a sugar, such as a dextran, or a polyvinyl alcohol or a poly(acrylic acid) or a poly(lactic-co-glycolic acid)).

8. A carrier system according to any preceding claim, wherein the carrier comprises a magnetic material.

9. A carrier system according to claim 8, wherein the carrier comprises a layered structure between a top surface of the carrier and an opposed bottom surface of the carrier, the layers including one or more magnetized layers.

10. A carrier system according to claim 9, in which the ratio of a lateral dimension of the one or more magnetized layers to a thickness or aggregate thickness of the magnetized layer or layers is greater than 500.

11. A carrier system according to any preceding claim, in which a minimum lateral dimension of the carrier is between 5 micrometres and 200 micrometres.

12. A carrier system according to any preceding claim, wherein the carrier is lithographically defined.

13. A carrier system according to any preceding claim, wherein the carrier comprises a photoresist layer, such as an SU-8 photoresist.

14. A carrier system according to any preceding claim, wherein a surface of the carrier is adapted for receiving the assay sample, for example wherein the surface comprises a gold cap layer on to which a polymer is covalently bonded by thiol group.

15. A carrier system according to any preceding claim, wherein the carrier comprises readable information, such as a readable code selected from a barcode or a 2D code.

16. A carrier system according to any preceding claim, comprising a plurality of carriers, wherein each of the carriers is secured to the substrate by the release layer.

17. A carrier system according to any preceding claim, comprising sterile packaging from which the carrier secured to the substrate is removable for use.

18. A method of manufacturing a carrier system for an assay, the method comprising the steps of:
providing a substrate;
forming a release layer on the substrate; and
depositing a carrier for receiving an assay sample on the release layer such that the carrier is secured to the substrate,
wherein the release layer is configured in use to release the carrier from the substrate in the presence of a biocompatible aqueous solution.

19. A method according to claim 18, wherein the step of forming the release layer on the substrate comprises spin-coating the release layer.

20. A method according to claim 18 or 19, wherein the step of depositing the carrier on the release layer comprises fabricating the carrier on the release layer, for example by a lithographic process.

21. A method according to any of claims 18 to 20, wherein the release layer is adapted to release the carrier from the substrate, in use, in the presence of the biocompatible aqueous solution within a time of between 1 hour to 72 hours.

22. A carrier system according to any of claims 18 to 21, in which the assay sample is receivable by the carrier while the carrier is in contact with the substrate.

23. A method according to any of claims 18 to 22, further comprising the step of adapting a surface of the carrier such that the surface is suitable for receiving an assay sample.

24. A method according to any of claims 18 to 23, further comprising the step of sterilizing the carrier system by immersing the carrier system in ethanol, following the step of depositing the carrier on the release layer.

25. A method according to any of claims 18 to 24, further comprising the step of packaging the carrier system within sterile packaging, with the carrier secured to the substrate.

26. A method according to any of claims 18 to 25, wherein the step of depositing the carrier comprises forming a magnetic structure.

27. A method of performing an assay using the carrier system as defined in any of claims 1 to 17, the method comprising the step of introducing a biocompatible aqueous solution to the carrier system to release the carrier.

28. A method of performing an assay according to claim 27, further comprising the step of introducing a sample for an assay to the carrier such that the sample is received by the carrier.

29. A method of performing an assay according to claim 28, wherein the sample is received by the carrier while the carrier is in contact with the substrate.

30. A method of performing an assay according to any of claims 27 to 29, in which the assay sample is suspended in the biocompatible aqueous solution.

31. A method of performing an assay according to any of claims 27 to 30, wherein the carrier comprises a magnetic material and the method further comprises applying a magnetic field to the carrier, the magnetic field acting to retain the carrier in contact with the substrate even after the release layer has released the carrier from the substrate.

32. A method of performing an assay according to claim 31, wherein the magnetic field is applied to retain the carrier in contact with the substrate for at least five seconds, or at least one minute, or at least 5 minutes or at least 30 minutes.

33. A method of using the carrier system as defined in any of claims 1 to 17, comprising the steps of introducing a sample for an assay to a carrier such that the sample is received by the carrier while the carrier is in contact with the substrate, and storing the sample received on the carrier.

34. A method according to claim 33, including the step of freezing the sample received on the carrier for storage.

35. A method as defined in claim 33 or 34, further comprising the step of releasing the carrier from the substrate before storing the sample received on the carrier.

36. A method for carrying out an assay, comprising the steps of;
providing a carrier secured to a substrate by a release layer which is configured to release the carrier from the substrate in the presence of a biocompatible aqueous solution; contacting the carrier secured to the substrate with a biocompatible solution in which an the assay sample is suspended;
receiving the assay sample onto the carrier while the carrier is in contact with the substrate; and releasing the carrier from the substrate by activation of the release layer by the biocompatible solution.

37. A carrier system or a method according to any preceding claim, in which the assay sample comprises a cell, preferably an adherent cell.
